(19) United States
(12) Patent Application Publication
(10) Pub. No.: US 2023/0348577 A1
(43) Pub. Date: Nov. 2, 2023

(54) MODIFIED FC REGIONS

(71) Applicant: adivo GmbH, Planegg (DE)

(72) Inventors: Verena BRAND, München (DE); Christina SCHNEIDER, München (DE); Andrea STERNER, Gröbenzell (DE); Markus WALDHUBER, München (DE)

(73) Assignee: adivo GmbH, Planegg (DE)

(21) Appl. No.: 17/800,630

(22) PCT Filed: Feb. 18, 2021
(86) PCT No.: PCT/EP2021/054057

§ 371 Date: Aug. 18, 2022

(30) Foreign Application Priority Data
Feb. 19, 2020 (EP) 20158132.9

(51) Int. Cl. C07K 16/18

(52) U.S. Cl. CPC C07K 16/18; C07K 2317/92; C07K 2317/71; C07K 2317/732; C07K 2317/734; C07K 2317/52

(57) ABSTRACT

The present invention relates to the field of modified constant domains of canine or feline antibodies having altered immune-effector functions and their use. More specifically, the application relates to modified Fc fragments having significantly reduced FcγRI and C1q binding.

### FIELD OF INVENTION

The present invention relates to the field of modified constant domains of canine or feline antibodies having altered immune-effector functions and their use. More specifically, the application relates to modified Fc fragments having significantly reduced FcγRI and C1q binding.

The accompanying sequence listing (in txt.-format) forms part of the disclosure content of the present application.

### BACKGROUND

Immunoglobulin G or IgG antibodies are large tetrameric proteins. Each IgG protein is composed of two identical light chains and two identical heavy chains which are linked to each other by disulfide bonds. There exist two types of light chains, referred to as kappa and lambda chains. Each of light chains is composed of one variable domain (VL) and one constant domain (CL). Also the heavy chains consist of one variable domain (VH) and three constant domains referred to as CH1, CH2, and CH3. A highly flexible amino acid stretch in the central part of the heavy chains, the so called “hinge region” links the CH1 and the CH2 domain.

In principle, an antibody can be segregated into two separate subunits: The “Fab” fragment, consisting of the light chain together with the VH and CH1 domains of the heavy chain, and the “Fc” that contains the remainder domains CH2 and CH3 of the heavy chain. Whereas the Fab fragment is responsible for antigen recognition and binding, the Fc interacts with the immune system to mediate effector functions such as antibody dependent cellular cytotoxicity (ADCC), antibody-dependent phagocytosis (ADCP) and complement dependent cytotoxicity (CDC).

Immunoglobulin G antibodies (IgG) from humans have been extensively studied and four human IgG subclasses, termed IgG1, IgG2, IgG3 and IgG4, have been described based on biological functions, biochemical properties and DNA sequences (Davies D R, Metzger H. Structural basis of antibody function. Annu Rev Immunol. 1983; 1:87-117; Jefferis R, Lund J, Goodall M. Recognition sites on human IgG for Fc gamma receptors: the role of glycosylation. Immunol Lett. 1995; 44(2-3):111-117; Shakib F. The human IgG subclasses. 1990. Pergamon Press, New York; Kenneth Murphy P T, Walport M). Each subclass has distinct characteristics and engages the immune system differently which is mediated by different binding affinities for immune effector proteins including the complement protein C1q, Fc gamma receptors (FcγRs) and the neonatal Fc receptor (FcRn). These interaction partners play crucial roles for complement-dependent cytotoxicity (CDC), antibody-dependent cell-mediated cytotoxicity (ADCC) and serum half-life, respectively.

In humans, complement activation is triggered most effectively by IgG1 and IgG3 (Bruggemann M, Williams G T, Bindon C I, et al. Comparison of the effector functions of human immunoglobulins using a matched set of chimeric antibodies. J Exp Med. 1987; 166(5):1351-1361; Michaelsen T E, Aase A, Westby C, Sandlie I. Enhancement of complement activation and cytolysis of human IgG3 by deletion of hinge exons. Scand J Immunol. 1990; 32(5):517-528). Binding of the antibody constant domain (Fc) to C1q, the first protein in the complement cascade, initiates complement helping to activate phagocytes and destroy pathogens (Schifferli J A, Ng Y C, Peters D K. The role of complement and its receptor in the elimination of immune complexes. N Engl J Med. 1986; 315(8):488-495; Garred P, Michaelsen T E, Aase A. The IgG subclass pattern of complement activation depends on epitope density and antibody and complement concentration. Scand J Immunol. 1989; 30(3):379-382; Moore G L, Chen H, Karki S, Lazar G A. Engineered Fc variant antibodies with enhanced ability to recruit complement and mediate effector functions. MAbs. 2010; 2(2):181-189). The ability of human IgG subclasses to trigger the ADCC by blood mononuclear cells has been shown to be strongest for IgG1 and IgG3. The high affinity of these subclasses to FcγRI and FcγRIII is associated with ADCC activity. In contrast, binding of other subclasses to the inhibitory receptor, FcγRIIb, contributes to lower ADCC activity (Daëron M. Fc receptor biology. Annu Rev Immunol. 1997; 15:203-234; Armour K L, Clark M R, Hadley A G, Williamson L M. Recombinant human IgG molecules lacking Fc gamma receptor I binding and monocyte triggering activities. Eur J Immunol. 1999; 29(8):2613-2624; 2-J; Clynes R A, Towers T L, Presta L G, Ravetch J V. Inhibitory Fc receptors modulate in vivo cytotoxicity against tumor targets. Nat Med. 2000; 6(4):443-446). Antibody binding to the FcRn on epithelial cells is associated with antibody recycling and correlative with serum half-life (Ghetie V, Hubbard J G, Kim J K, Tsen M F, Lee Y, Ward E S. Abnormally short serum half-lives of IgG in beta 2-microglobulin-deficient mice. Eur J Immunol. 1996; 26(3):690-696, Wilsker D F, Hayes K C, Schoenfeld D, Simister N E. Increased clearance of IgG in mice that lack beta 2-microglobulin: possible protective role of FcRn. Immunology. 1996; 89(4):573-578; Praetor A, Hunziker W. beta(2)-Microglobulin is important for cell surface expression and pH-dependent IgG binding of human FcRn. J Cell Sci. 2002; 115(Pt 11):2389-2397; Jefferis R. Antibody therapeutics: isotype and glycoform selection. Expert Opin Biol Ther. 2007; 7(9):1401-1413).

In addition to human IgGs, immunoglobulin classes of rodents (mice and rats) have been well characterized. In contrast, less is known about the functional properties of IgG subclasses of companion animals such as dogs or cats.

Canine IgGs consist of four subclasses, referred to as calgG-A (HC-A), calgG-B (HC-B), calgG-C (HC-C) and calgG-D (HC-D) (Tang L, Sampson C, Dreitz M J, McCall C. Cloning and characterization of cDNAs encoding four different canine immunoglobulin gamma chains. Vet Immunol Immunopathol. 2001; 80(3-4):259-270). In addition, canine FcγRs analogous to human receptors I, IIA, IIB, and III have been described (Nimmerjahn F, Ravetch J V. Fc gamma receptors: old friends and new family members. Immunity. 2006; 24(1):19-28), although the inhibitory canine FcγRIIA could not be confirmed in another study (Bergeron L M, McCandless E E, Dunham S, et al. Comparative functional characterization of canine IgG subclasses. Vet Immunol Immunopathol. 2014; 157(1-2):31-41). In vitro binding experiments revealed that canine HC-B and HC-C strongly bind to canine FcγRs whereas HC-A only binds weakly (Bergeron, 2013). Similarly, HC-B and HC-C were reported to bind tightly to human C1q protein, whereas HC-A and HC-D bind with little to no affinity (Bergeron, 2013). With the exception of HC-C, all canine subclasses strongly bind to the FcRn (Bergeron, 2013).

Antibody purification strategies typically include a Staphylococcus Protein A affinity chromatography step. Of the four canine subclasses, only HC-B has been reported to bind strongly to Protein A. HC-A has weak affinity to Staphylococcus Protein A and both HC-C and HC-D subclasses do not bind. However, using Streptococcus Protein G resins, all four canine subclasses can be purified (Bergeron, 2013).

Depending on the therapeutic application, the selection of the respective antibody subclass is crucial and one needs to consider whether engagement of humoral or cellular components of the immune system is advantageous or even might lead to unwanted side effects of a drug. For example, a therapeutic antibody against tumor cell growth or a pathogen should have strong effector functions. In contrast, targeting soluble mediators or cell surface receptors of a healthy cell to prevent receptor-ligand interactions typically requires absence of any CDC or ADCC activity to prevent target cell death or unwanted cytokine secretion. Disease areas in which silent antibody formats are necessary contain but are not limited to inflammatory diseases (e.g. rheumatoid arthritis, psoriasis, inflammatory bowel disease), allergies (e.g. asthma), pain (e.g. osteoarthritic pain, cancer pain, lower back pain) and eye disease (e.g. age related macular degeneration).

It is well known that IgG antibodies mediate effector functions such as ADCC through binding of their Fc portion to the family Fc-receptors, whereas CDC is mediated through the binding of the Fc to the first component of complement, C1q. Enhancement or elimination of effector functions can be achieved through mutations in the Fc portion of an antibody which alter the affinity to respective interaction molecules. There are numerous reports in the prior art describing amino acid substitutions that may be introduced into an antibody molecule in order to modulate its effector functions. For example, an asparagine to alanine (N297A) substitution in a human IgG1, which results in a non-glycosylated antibody, significantly reduces antibody binding to several Fc-receptors (Shields R L, Namenuk A K, Hong K, et al. High resolution mapping of the binding site on human IgG1 for Fc gamma RI, Fc gamma RII, Fc gamma RIII, and FcRn and design of IgG1 variants with improved binding to the Fc gamma R. J Biol Chem. 2001; 276(9):6591-6604). Additionally, an aspartic acid-to-alanine (D265A) substitution also significantly reduces binding of the antibody to Fc receptors. Each of the N297A and D265A substitutions were also shown to significantly impair CDC (Shields 2001). There are other similar reports identifying potential substitutions to reduce or eliminate effector function in antibodies (e.g., Xu D, Alegre M L, Varga S S, et al. In vitro characterization of five humanized OKT3 effector function variant antibodies. Cell Immunol. 2000; 200(1):16-26; Alegre M L, Collins A M, Pulito V L, et al. Effect of a single amino acid mutation on the activating and immunosuppressive properties of a “humanized” OKT3 monoclonal antibody. J Immunol. 1992; 148(11):3461-3468; Bolt S, Routledge E, Lloyd I, et al. The generation of a humanized, non-mitogenic CD3 monoclonal antibody which retains in vitro immunosuppressive properties. Eur J Immunol. 1993; 23(2):403-411; Tao M H, Morrison S L. Studies of aglycosylated chimeric mouse-human IgG. Role of carbohydrate in the structure and effector functions mediated by the human IgG constant region. J Immunol. 1989; 143(8):2595-2601; Walker M R, Lund J, Thompson K M, Jefferis R. Aglycosylation of human IgG1 and IgG3 monoclonal antibodies can eliminate recognition by human cells expressing Fc gamma RI and/or Fc gamma RII receptors. Biochem J. 1989; 259(2):347-353).

The N297 residue is not only conserved in humans but in the whole class of mammals, specifically dog, cat, bovine, camel, horse, macaques, monkeys, opossum, mouse, rabbit, sheep, chimpanzee, rat, and pig. It is generally known that a strong conservation of residues over a large number of species is phenotypically linked with a conserved function of the respective residue and introducing a N297A mutation in any of the other species will likely reduce immune effector function as demonstrated in human.

Accordingly, EP 2 705 057 A1 discloses non-glycosylated canine antibodies generated by the introduction of an asparagine to alanine (N297A) substitution in canine HC-B and HC-C. The variants are indeed characterized by an abolished or diminished binding to C1q. However, aglycosylation may negatively impact the plasma half live of antibodies as shown by Chen at al. (Chen T F, Sazinsky S L, Houde D, et al. Engineering Aglycosylated IgG Variants with Wild-Type or Improved Binding Affinity to Human Fc Gamma RIIA and Fc Gamma RIIIAs. J Mol Biol. 2017; 429(16):2528-2541), and may thus require higher doses or more frequent administration of a recombinant antibody. Furthermore, aglycosylation may decrease thermostability (Ghirlando R, Lund J, Goodall M, Jefferis R. Glycosylation of human IgG-Fc: influences on structure revealed by differential scanning micro-calorimetry. Immunol Lett. 1999; 68(1):47-52.) and increased susceptibility to proteolysis (Raju T S, Scallon B J. Glycosylation in the Fc domain of IgG increases resistance to proteolytic cleavage by papain. Biochem Biophys Res Commun. 2006; 341(3):797-803.).

Also, the HC-A and HC-D subtypes were reported not to bind C1q and did not result in complement activation and potentially other downstream effector functions, such as ADCC and ADCP.

Although canine antibodies of the HC-A and HC-D isotypes have desirable lack of binding to complement for applications where target neutralization is not required, these only bind weakly to Staphylococcus Protein A, making the development of commercially viable manufacturing and purification methods more complex. In contrast, aglycosylated canine HC-B antibodies retain binding to Staphylococcus Protein A, rendering this variant the more suitable candidate lacking effector functions. For human antibodies, the aglycosylation approach has proven successful in abrogating binding to low affinity FcγRs and effector functions such as CDC and ADCC. However, it was also recognized that under avidity based binding conditions, effector functions can be retained (Lo M, Kim H S, Tong R K, et al. Effector-attenuating Substitutions That Maintain Antibody Stability and Reduce Toxicity in Mice. J Biol Chem. 2017; 292(9):3900-3908; Nesspor T C, Raju T S, Chin C N, Vafa O, Brezski R J. Avidity confers FcγR binding and immune effector function to aglycosylated immunoglobulin G1. J Mol Recognit. 2012; 25(3):147-154). Moreover, as disclosed in another study (WO 20151091910 A2) a single substitution at position N297A in canine HC-B that is analogous to that of human IgG1 and results in an aglycosylated antibody, does not completely eliminate both ADCC and CDC effector functions in the corresponding canine antibody.

To develop a “silent” canine therapeutic antibody format, none of the naturally occurring canine IgG subclasses as well as the aglycosylated HC-B variant satisfy all required properties, i.e. lack of effector functions such as antibody-dependent cytotoxicity (ADCC), antibody-dependent phagocytosis (ADCP) and complement-dependent cytotoxicity (CDC), long in vivo half-life and the possibility to be purified by industry standard technologies such as Protein A chromatography.

Further to the above mentioned N297A mutants, some other silencing mutants are known in the prior art.

WO 2018/073185 A1 discloses that mutations in residues 253, 255, 257 may increase FcRn binding of constant regions. However, no effect on ADCC or CDC was shown.

WO 2019/035010 A1 speculates that mutations in residues 5, 38, 38, 97, 98, which were identified after analysis of the protein sequence and 3-D structure modelling of canine IgG-B and IgG-C compared to IgG-A and IgG-D, may impact on ADCC activity. However, this assumption was not confirmed experimentally.

WO 2015/091910 A2 discloses that mutation in residues 4, 31, 63, 93 and 95 reduce C1q and FcγRI binding. However, it was not shown that binding to FcRn is not likewise decreased.

Very little is known about the functional properties of feline IgGs. Two allelic sequences referred to as feline IgG1a and 1b have been described which function similar to human IgG1 and are expected to induce strong effector function in vivo (Strietzel C J, Bergeron L M, Oliphant T, Mutchler V T, Choromanski L J, Bainbridge G. In vitro functional characterization of feline IgGs. Vet Immunol Immunopathol. 2014; 158(3-4):214-223). The same authors report the presence of a rare IgG sequence, now referred to as feline IgG2. This additional IgG does not bind to recombinant fFcγRI or fFcγRIII and has negligible binding to hC1q indicative of lack of effector function.

The problem underlying the present invention was the provision of canine and feline Fc fragments of antibodies with augmented, decreased, or eliminated binding to C1q and FcγRs that overcome the drawbacks of Fc fragments known in the art.

### SUMMARY OF INVENTION

The problem underlying the invention is solved by the polypeptide and methods according to the appended claims and as further described herein.

The present invention solves the problem by providing a polypeptide comprising at least a canine or feline Fc fragment, wherein the Fc fragment comprises at least one substitution of an amino acid selected from at least one of amino acid positions 235, 239, 270, and/or 331 relative to the wild type Fc fragment. Preferably the Fc fragment is from isotype B of canine IgG.

In a preferred embodiment the present invention relates to a polypeptide comprising at least a canine or feline Fc fragment, wherein the Fc fragment comprises least two substitutions of amino acids selected from at least two of the amino acids at positions 234, 235, 239, 270, and/or 331. More preferably, the two amino acids are 235 and 239; 235 and 270; 235 and 331; 239 and 270; 239 and 331; 270 and 331, 234 and 235, 234 and 239; 234 and 270; or 234 and 331.

In another preferred embodiment the present invention relates to a polypeptide comprising at least a canine or feline Fc fragment, wherein the Fc fragment comprises least three substitutions of amino acids selected from at least three of amino acid positions 234, 235, 239, 270, and/or 331. More preferably, the three amino acid positions are 235, 239, and 270; 239, 270, and 331; 235, 270, and 331; or 235, 239, and 331.

In another preferred embodiment the present invention relates to a polypeptide comprising at least a canine or feline Fc fragment, wherein the Fc fragment comprises at least four substitutions of amino acids selected from amino acid positions 234, 235, 239, 270, and 331, more preferably 235, 239, 270, and 331, and most preferably amino acids L235, S239, D270, and P331.

The polypeptide according to the present invention may comprise SEQ ID NO: 8 to SEQ ID NO: 29, more preferably SEQ ID NO: 18, 19, 26, 27, or 29. Most preferably the polypeptide comprises SEQ ID NO: 19 or 27.

Surprisingly, the polypeptides according to the invention exhibit a reduced binding affinity to C1q and/or an Fc receptor relative to a polypeptide comprising the corresponding wild type Fc fragment. Under physiological conditions of an uncompromised immune system, reduced binding or diminished binding to C1q and/or FcγRI results in a reduction or complete elimination of the immune effector functions of the complement-dependent cytotoxicity (CDC) and induction of antibody-dependent cytotoxicity (ADCC). The reduced binding or diminished binding of polypeptides comprising at least one substitution in the Fc fragment to C1q and/or FcγRI and/or the resulting reduction or complete elimination of CDC or ADCC is also referred to as “silencing” herein.

In an embodiment of the present invention, preferably wherein the polypeptide is from isotype B of canine IgG, the Fc fragment surprisingly maintains its ability to bind to neonatal Fc receptor (FcRn) as well as to Protein A.

As shown in FIG. 4, the polypeptides comprising a mutated Fc fragment of the HC-B isotype according to the present invention maintain their ability to bind to neonatal Fc receptor (FcRn).

The invention will be described in more detail in the following

### DETAILED DESCRIPTION

In a first aspect, the present invention relates to a polypeptide comprising at least a canine or feline Fc fragment, wherein the Fc region comprises at least one substitution of an amino acid selected from at least one of amino acid positions 235, 239, 270, and/or 331 relative to the wild type Fc region. Preferably the Fc region is from isotype B of canine IgG.

Unless explicitly described otherwise for specific embodiments the “amino acid position” referred to herein is the number of the position of an amino acid according to the EU numbering system (Kabat et al., Sequences of Proteins of Immunological Interest, 5th Ed. Public Health Service, National Institutes of Health, Bethesda, Md. (1991)). Woof et al. Molec. Immunol. 23:319-330 (1986); Duncan et al. Nature 332:563 (1988); Canfield and Morrison, J. Exp. Med. 173:1483-1491 (1991); Chappel et al., Proc. Natl. Acad. Sci USA 88:9036-9040 (1991) when the canine or feline Fc regions are aligned to a human IgG1. The number of the position in the canine or feline Fc region corresponds to the number of the position in the aligned human IgG. As shown in FIG. 6 a&b, the alignment of the canine and feline sequences to the human sequence results in gaps in the canine and feline sequences. Accordingly, some sequences do not have an amino acid at every digit of the consecutive numbering. While the EU numbering system was originally applied to human IgG1 antibodies, it is also applied to antibodies from other species. The number of the position of an amino acid in an antibody or antibody derived molecule according to the EU system can easily be transferred into alterative amino acid numbering systems for antibodies such as the numbering system according to Chothia (Chothia C & Lesk A M (1987) Canonical structures for the hypervariable regions of immunoglobulins. J Mol Biol. 196(4):901-17; Chothia C, Lesk A M, Tramontano A, Levitt M, Smith-Gill S J, Air G, Sheriff S, Padlan E A, Davies D, Tulip W R (1989) Conformations of immunoglobulin hypervariable regions. Nature. 342(6252):877-83) or IMGT (Lefranc M P, Giudicelli V, Ginestoux C, Bodmer J, Müller W, Bontrop R, Lemaitre M, Malik A, Barbie V, Chaume D (1999) IMGT, the international ImMunoGeneTics database. Nucleic Acids Res. 27(1):209-12). FIG. 6 shows canine and feline Fc wild-type sequences with the numbering according to EU system.

The number of the position of an amino acid according to this invention may also be assigned according to the position in the alignment as shown in FIG. 2, page 266, of Tang et al (Tang L, Sampson C, Dreitz M J, McCall C (2001) Cloning and characterization of cDNAs encoding four different canine immunoglobulin gamma chains. Vet Immunol Immunopathol. 80 (3-4):259-70). It should be noted that the numbering of the positions according to Tang et al. also includes the gaps in the assignment and does not start with the first amino acid of the Fc region but, with the N-terminus of a full canine antibody including CDRs and leader sequence. In this alternative numbering according to Tang et al. amino acid position numbers 262, 263, 267, 298, and 359 correspond to 234, 235, 239, 270, and 331 respectively of the EU numbering.

The term “Fc fragment” relates to a fragment of an immunoglobulin comprising at least parts of, or the entire constant heavy chain region 2 (C2 or CH2) and constant heavy chain region 3 (C3 or CH3) or of the crystallisable fragment of an immunoglobulin obtained by papain digestion. The fragment is understood to be a part of a larger polypeptide sequence. Thus the “fragment” will usually have amino acids sequence bound to the C- and/or N-terminus. The terms “C2” or “CH2” as well as terms “C3” or “CH3” may be used interchangeably. Furthermore, the terms “Fc region” and “Fc domain” may be used interchangeably when referring to the immunoglobulin Fc C H2 and CH3 sequences unless explicitly stated otherwise. Within the context of the present invention, the boundaries of the CH2 and CH3 region for canine immunoglobulin isotypes HC-A, HC-B, HC-C and HC-D are defined according to Tang et al. (Tang L, Sampson C, Dreitz M J, McCall C (2001) Cloning and characterization of cDNAs encoding four different canine immunoglobulin gamma chains. Vet Immunol Immunopathol. 80 (3-4):259-70), which is incorporated herein by reference.

The Fc region according to the present in invention is an Fc region from dog, thus a canine Fc region, or from cat, thus a feline Fc region.

The terms “dog” or “canine” refer to all domestic dogs, Canis lupus familiaris or Canis familiaris. Likewise, the terms “cat” or “feline” refer to domestic cats, Felis catus, Felis catus domesticus Felus angorensis, and Felis vulgaris.

The Fc region according to the present invention comprises at least one substitution of an amino acid relative to the wild type Fc region. The term “substitution” refers to the replacement of an amino acid in a sequence by at least another amino acid, preferably with one amino acid. The polypeptides of the invention may comprise one, two, three, four, five, six or more amino substitutions.

Within the context of the present invention, a “wild type” Fc region is a Fc region having a naturally occurring amino acid sequence which has not been artificially rendered, for example by introducing mutations by methods of genetic engineering. The wild type sequence of an Fc region according to the present invention comprising at least one substitution of an amino acid relative to the wild type Fc region is also referred to as “corresponding wild type” or “corresponding wild type sequence” herein. An Fc region comprising at least one substitution of an amino acid relative to the “wild type” is also referred to as “mutant” within the context of the present invention.

The Fc region according to the present invention may be selected from canine isotype A of immunoglobulin G (also termed HC-A, HCA, calgG-A), isotype B of immunoglobulin G (also termed HC-B, HCB, calgG-B), isotype C of immunoglobulin G (also termed HC-C, HCC, calgG-C), or isotype D of immunoglobulin G (also termed HC-D, HCD, calgG-D). Preferably the Fc region is selected from isotype B. Feline Fc regions may be from immunoglobulin G isotype 1a (also termed IgG1a), isotype 1b (also termed IgG1b), and isotype 2 (also termed IgG2).

In a specific embodiment, the canine wild type sequences referred to herein are the sequences according to SEQ ID NO: 1 to 4 discloses in FIG. 6 a&b and Table 1a.

TABLE 1a
Canine wild type sequences

| SEQ | | |
|---|---|---|
| ID | canine | |
| NO: | isotype | |
| 1 | HC-A | ASTTAPSVFPLAPSCGSTSGSTVALACLVSGYFPEPVTVSWNSGSLTSGVH |
| | | TFPSVLQSSGLHSLSSMVTVPSSRWPSETFTCNVVHPASNTKVDKPVFNE |
| | | CRCTDTPPCPVPEPLGGPSVLIFPPKPKDILRITRTPEVTCVVLDLGREDPEV |
| | | QISWFVDGKEVHTAKTQSREQQFNGTYRVVSVLPIEHQDWLTGKEFKCRV |
| | | NHIDLPSPIERTISKARGRAHKPSVYVLPPSPKELSSSDTVSITCLIKDFYPPD |
| | | IDVEWQSNGQQEPERKHRMTPPQLDEDGSYFLYSKLSVDKSRWQQGDPF |
| | | TCAVMHETLQNHYTDLSLSHSPGK |
| 2 | HC-B | ASTTAPSVFPLAPSCGSTSGSTVALACLVSGYFPEPVTVSWNSGSLTSGVH |
| | | TFPSVLQSSGLYSLSSMVTVPSSRWPSETFTCNVAHPASKTKVDKPVPKRE |
| | | NGRVPRPPDCPKCPAPEMLGGPSVFIFPPKPKDTLLIARTPEVTCVVVDLDP |
| | | EDPEVQISWFVDGKQMQTAKTQPREEQFNGTYRVVSVLPIGHQDWLKGKQ |
| | | FTCKVNNKALPSPIERTISKARGQAHQPSVYVLPPSREELSKNTVSLTCLIKD |
| | | FFPPDIDVEWQSNGQQEPESKYRTTPPQLDEDGSYFLYSKLSVDKSRWQR |
| | | GDTFICAVMHEALHNHYTQESLSHSPGK |
| 3 | HC-C | ASTTAPSVFPLAPSCGSQSGSTVALACLVSGYIPEPVTVSWNSVSLTSGVH |
| | | TFPSVLQSSGLYSLSSMVTVPSSRWPSETFTCNVAHPATNTKVDKPVAKEC |
| | | ECKCNCNNCPCPGCGLLGGPSVFIFPPKPKDILVTARTPTVTCVVVDLDPEN |
| | | PEVQISWFVDSKQVQTANTQPREEQSNGTYRVVSVLPIGHQDWLSGKQFK |
| | | CKVNNKALPSPIEEIISKTPGQAHQPNVYVLPPSRDEMSKNTVTLTCLVKDFF |
| | | PPEIDVEWQSNGQQEPESKYRMTPPQLDEDGSYFLYSKLSVDKSRWQRG |
| | | DTFICAVMHEALHNHYTQISLSHSPGK |
| 4 | HC-D | ASTTAPSVFPLAPSCGSTSGSTVALACLVSGYFPEPVTVSWNSGSLTSGVH |
| | | TFPSVLQSSGLYSLSSTVTVPSSRWPSETFTCNVVHPASNTKVDKPVPKES |
| | | TCKCISPCPVPESLGGPSVFIFPPKPKDILRITRTPEITCVVLDLGREDPEVQI |
| | | SWFVDGKEVHTAKTQPREQQFNSTYRVVSVLPIEHQDWLTGKEFKCRVNHI |
| | | GLPSPIERTISKARGQAHQPSVYVLPPSPKELSSSDTVTLTCLIKDFYPPEIDV |
| | | EWQSNGQPEPESKYHTTAPQLDEDGSYFLYSKLSVDKSRWQQGDTFTCA |
| | | VMHEALQNHYTDLSLSHSPGK |

The underlined sequence from position 234 to 331 depicts the sequence range containing substitutions as described before.

Alternatively, canine wild-type sequences according to the present invention are published elsewhere:

TABLE 1b
Canine wild type sequences

| canine | | |
|---|---|---|
| isotype | Disclosure | |
| HC-A | GeneBank accession No. AF354264 | |
| | (also disclosed by Tang et al. 2001); | |
| | WO2012153126 A1 ID NO: 8 | |
| HC-B | GeneBank accession No. AF354265 | A206P substitution |
| | (also disclosed by Tang et al. 2001) | in comparison to |
| | | SEQ ID NO: 2 |
| | WO 2010/027488 A2, SEQ ID NO: 54 | E439K substitution |
| | | in comparison to |
| | | SEQ ID NO: 2 |
| | WO 2012/153126 A1, SEQ ID NO: 9 | F373Y substitution |
| | | in comparison to |
| | | SEQ ID NO: 2 |
| HC-C | GeneBank accession No. AF354266 | |
| | (also disclosed by Tang et al. 2001) | |
| | WO 2012/153126 A1, SEQ ID NO: 10 | |
| HC-D | GeneBank accession No. AF354267 | |
| | (also disclosed by Tang et al. 2001) | |
| | WO 2012/153126 A1, SEQ ID NO: 11 | |

In a specific embodiment, the feline wild type sequences referred to herein are the sequences according to SEQ ID NO: 5 to 7 shown in Table 2 and in FIG. 6 a&b.

TABLE 2
feline wild type sequences

| SEQ | | |
|---|---|---|
| ID | feline | |
| NO: | isotype | |
| 5 | IgG1a | ASTTAPSVFPLAPSCGTTSGATVALACLVLGYFPEPVTVSWNSG |
| | | ALTSGVHTFPAVLQASGLYSLSSMVTVPSSRWLSDTFTCNVAHP |
| | | PSNTKVDKTVRKTDHPPGPKPCDCPKCPPPEMLGGPSIFIFPPK |
| | | PKDTLSISRTPEVTCLVVDLGPDDSDVQITWFVDNTQVYTAKTSP |
| | | REEQFNSTYRVVSVLPILHQDWLKGKEFKCKVNSKSLPSPIERTI |
| | | SKAKGQPHEPQVYVLPPAQEELSENKVSVTCLIKSFHPPDIAVE |
| | | WEITGQPEPENNYRTTPPQLDSDGTYFVYSKLSVDRSHWQRGN |
| | | TYTCSVSHEALHSHHTQKSLTQSPGK |
| 6 | IgG1b | ASTTAPSVFPLAPSCGTTSGATVALACLVLGYFPEPVTVSWNS |
| | | GALTSGVHTFPAVLQASGLYSLSSMVTVPSSRWLSDTFTCNVA |
| | | HPPSNTKVDKTVRKTDHPPGPKPCDCPKCPPPEMLGGPSIFIFP |
| | | PKPKDTLSISRTPEVTCLVVDLGPDDSDVQITWFVDNTQVYTAK |
| | | TSPREEQFNSTYRVVSVLPILHQDWLKGKEFKCKVNSKSLPSPI |
| | | ERTISKDKGQPHEPQVYVLPPAQEELSENKVSVTCLIEGFYPSDI |
| | | AVEWEITGQPEPENNYRTTPPQLDSDGTYFLYSRLSVDRSRWQ |
| | | RGNTYTCSVSHEALHSHHTQKSLTQSPGK |
| 7 | IgG2 | ASTTAPSVFPLAPSCGTTSGATVALACLVLGYFPEPVTVSWN |
| | | SGALTSGVHTFPAVLQASGLYSLSSMVTVPSSRWLSDTFTCN |
| | | VAHPPSNTKVDKTVPKTASTIESKTGEGPKCPVPEIPGAPSVF |
| | | IFPPKPKDTLSISRTPEVTCLVVDLGPDDSNVQITWFVDNTEM |
| | | HTAKTRPREEQFNSTYRVVSVLPILHQDWLKGKEFKCKVNSK |
| | | SLPSAMERTISKAKGQPHEPVYVLPPTQEELSENKVSVTCLIK |
| | | GFHPPDIAVEWEITGQPEPENNYQTTPPQLDSDGTYFLYSRL |
| | | SVDRSHWQRGNTYTCSVSHEALHSHHTQKSLTQSPGK |

Alternatively, feline wild-type sequences according to the present invention are disclosed by Strietzel et al. (Strietzel C J, Bergeron L M, Oliphant T, Mutchler V T, Choromanski L J, Bainbridge G (2014) In vitro functional characterization of feline IgGs. Vet Immunol Immunopathol, 158(3-4):214-23), page 220.

In a preferred embodiment the polypeptide according to the present invention comprises at least a sequence corresponding to amino acids 234 to 331 according to Kabat numbering having at least the one substitution of an amino acid selected from at least one of amino acid position 235, 239, 270, and/or 331 relative to the wild type Fc region. The underscored sequence in Tables 1a and 2 corresponds to amino acids 234 to 331 according to Kabat numbering in canine or feline wild type Fc region sequences.

In summary, the wild type sequence according to the present invention may preferably be selected from any of Seq ID No: 1 to 7, the sequences of amino acids 234 to 331 (according to Kabat numbering) of GeneBank accession Nos. AF354264, AF354265, AF354266, AF354267, or the wild type sequences disclosed by Strietzel et al., page 220. Accordingly, the (amino acid) sequences (according to Kabat numbering) of GeneBank accession Nos. AF354264, AF354265, AF354266, AF354267, and the wild type sequences disclosed by Striezel et al., page 220, are explicitly incorporated herein as reference sequences, and thus from part of the disclosure content of the present application.

As further described herein, a substitution of an amino acid selected from at least one of amino acid positions 235, 239, 270, and/or 331 relative to the wild type Fc fragment may in various embodiments be described as an amino acid substitution in at least one of the (amino acid) positions corresponding to positions 235, 239, 270, and/or 331 of the (amino acid) sequence of the wild type Fc fragment. Accordingly, reference to at least one substitution of an amino acid selected from at least one of amino acid position 235, 239, 270, and/or 331 relative to the wild type Fc fragment may in various embodiments be described as at least one amino acid substitution in at least one of the (amino acid) positions corresponding to positions 235, 239, 270, and/or 331 of the (amino acid) sequence of the wild type Fc fragment.

Accordingly, reference to at least one substitution of an amino acid selected from at least one of amino acid position 235, 239, 270, and/or 331 relative to the wild type Fc fragment may in various embodiments be described as at least one amino acid substitution in at least one of the (amino acid) positions corresponding to positions 235, 239, 270, and/or 331 of the (amino acid) sequence of the wild type Fc fragment as disclosed in any of Seq ID NOs: 1 to 7, or the (amino acid) sequences (according to Kabat numbering) of GeneBank accession Nos. AF354264, AF354265, AF354266, AF354267, or the (wild type) sequences as disclosed by Striezel et al., page 220.

As further described herein, the terms “relative to the wild type Fc fragment (region)” and “relative to the amino acid sequence of the wild type Fc fragment (region)” may be used interchangeably herein.

According to the present invention, a polypeptide sequence having “a substitution of an amino acid relative to the wild type Fc region” at a specified position is a polypeptide characterized by an amino acid sequence having at least 96%, preferably 98%, more preferably 99%, and most preferably 100% identity to the wild type sequence referred to, except for the specified substitution. Accordingly, the polypeptide according to the present invention may also comprise mutations such as insertions, deletions of substitutions, other than the “substitution of at least one amino acid” as described herein.

The “percent (%) identity” with respect to a given amino acid sequence are defined within the context of the present invention as the percentage of amino acid residues in a reference sequence that are identical with the amino acid residues in the amino acid sequence compared to, after aligning the sequences and introducing gaps, if necessary to achieve the maximum percent sequence identity, and not considering any conservative substitutions as part of the sequence identity. Alignment for purposes of determining percent sequence identity within the present invention can be carried out in various ways well known to the person skilled in the art, for instance, using publicly available computer software such as BLAST, BLAST-2, ALIGN, or MEGALINE™ (DNASTAR) software. The person skilled in the art is routinely able to determine appropriate parameters for measuring alignment, including any algorithms needed to achieve maximal alignment over the full length of sequences being compared.

The amino acid substitutions may be conservative or non-conservative substitutions. Amino acids may be grouped according to common side-chain properties: (1) hydrophobic: Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. In non-conservative substitutions an amino acid of one group is exchanged with an amino acid from a different group. In a conservative substitution and amino acid of one group is exchanged with another amino acid from the same group.

Instead of substituting a natural amino acid comprised in a wild-type polypeptide sequence with another natural amino acid, the term amino acid substitution also encompasses the substitution of a natural amino acid with an amino acid derivative. “Amino acid derivative” as used herein refers to any non-natural amino acid, modified amino acid, and/or amino acid analogue not found in mammals. Exemplary amino acid derivatives include natural amino acids not found in humans (e.g., seleno cysteine and pyrrolysine, which may be found in some microorganisms) or chemically modified amino acids.

In a preferred embodiment, the present invention relates to a polypeptide comprising at least a canine or feline Fc fragment, wherein the Fc region comprises at least one substitution of an amino acid selected from at least one of L235, S239, D270, and/or P331 relative to the wild type Fc region.

As disclosed in FIG. 2, the inventors have surprisingly found, that any single of the mutations of L235, S239, D270, and/or P331 affects both C1q and FcγRI binding of the Fc fragment. These results are especially surprising, since mutations of S239, D270, or P331 in the human Fc do not impair the binding to the human FcγRI, as disclosed by Shields et al. (Shields R L, Namenuk A K, Hong K, et al. High resolution mapping of the binding site on human IgG1 for Fc gamma RI, Fc gamma RII, Fc gamma RIII, and FcRn and design of IgG1 variants with improved binding to the Fc gamma R. J Biol Chem. 2001; 276(9):6591-6604). Therefore, in a preferred embodiment the present invention relates to a polypeptide comprising at least a canine or feline Fc fragment, wherein the Fc region comprises at least one substitutions of an amino acids selected from the group of amino acids at positions 239, 270, and/or 331.

The observed difference in the effects of mutations in the canine Fc region versus the effect of the corresponding mutation in the human Fc region is also confirmed in Experiment 1 for the mutation M234A/L235A for which a significantly reduction of effector function has been described in the human system (Xu D, Alegre M L, Varga S S, et al. In vitro characterization of five humanized OKT3 effector function variant antibodies. Cell Immunol. 2000; 200(1):16-26.). In contrast thereto, the same mutations in the canine framework abolished FcγRI binding but the variant is still able to bind C1q.

In another preferred embodiment the present invention relates to a polypeptide comprising at least a canine or feline Fc fragment, wherein the Fc region comprises at least two substitutions of amino acids selected from at least two of the amino acids at positions 234, 235, 239, 270, and/or 331. Preferably, a substitutions of amino acids selected from at least two of the amino acids M234, L235, S239, D270, and/or P331, especially at least two amino acids selected from the group of amino acids consisting of S239, D270, or P331. More preferably, the two amino acids are the amino acids at positions 235 and 239; 235 and 270; 235 and 331; 239 and 270; 239 and 331; 270; 331, 234 and 235; 234 and 239; 234 and 270; 234 and 331; 234 and 331. More preferably, the two amino acids are L235 and S239; L235 and D270; L235 and P331; S239 and D270; S239 and P331; D270 and P331, M234 and L235; M234 and S239; M234 and D270; M234 and P331. Most preferably, the two amino acid positions are 235 and 331, respectively the amino acids L235 and P331. Specifically, the substitutions may be M234A and L235A; L235A and S239A; L235A and D270A; L235A and P331G; S239A and D270A; S239A and P331G; or D270A and P331G.

In another preferred embodiment the present invention relates to a polypeptide comprising at least a canine or feline Fc fragment, wherein the Fc region comprises at least two substitutions of amino acids selected from amino acid positions 234, 235, 239, 270, and/or 331 wherein at least one of the two amino acid positions is selected from 239, 270, and/or 331

In another preferred embodiment the present invention relates to a polypeptide comprising at least a canine or feline Fc fragment, wherein the Fc region comprises least three substitutions of amino acids selected from at least three of amino acid positions 234, 235, 239, 270, and/or 331. Preferably, a substitution of amino acids selected from at least M234, L235, S239, D270, and/or P331. More preferably, the three amino acid positions are 235, 239, and 270; 239, 270, and 331; 235, 270, and 331; or 235, 239, and 331. More preferably, the three amino acids are L235, S239, and D270; S239, D270, and P331; L235, D270, and P331; or L235, S239, and P331. Most preferably the three amino acid positions are 235, 239, and 270; or 235, 239, and 331, respectively the amino acids L235, S239 and D270; or L235, S239, and P331. Specifically, the substitutions may be L235A, S239A, and D270A; S239A, D270A, and P33G1; L235A, D270A, and P331G; or L235A, S239A, and P331G.

In another preferred embodiment the present invention relates to a polypeptide comprising at least a canine or feline Fc fragment, wherein the Fc region comprises at least four substitution selected from amino acid positions 234, 235, 239, 270, and 331. Preferably amino acids 235, 239, 270, and 331, more preferably from L235, S239, D270, and P331. Specifically, the substitutions may be L235A, S239A, D270A, and P331G.

In the afore described polypeptides, the one or more substitution preferably is a substitution of the wild type amino acid by alanine, glycine, glutamine, valine, or serine. More preferably, leucine, preferably L235 is substituted by alanine, glutamine or valine, most preferably alanine.

Serine, preferably S239, is preferably substituted by alanine or valine, most preferably alanine. Aspartate, preferably D270, is preferably substituted by alanine or valine, most preferably alanine. Proline, preferably P331, is preferably substituted by glycine, alanine or serine, most preferably by glycine.

Accordingly, the one or more substitution as described above is selected from L235A, S239A, D270A, and/or P331G.

In a preferred embodiment of the invention the polypeptides according to the invention comprise at least a canine Fc fragment form immunoglobulin isotype B.

As further described herein, the terms “Fc region” and “Fc domain” and “Fc fragment” may be used interchangeably. In particular, the terms “Fc region” and “Fc fragment” may be used interchangeably herein. More specifically, the terms “canine or feline Fc region” and “canine or feline Fc fragment” may be used interchangeably herein. The same applies with regard to the terms “wild type Fc region” and “wild type Fc fragment”, which may also be used interchangeably herein.

The polypeptide according to the present invention may comprise a sequence selected from SEQ ID NOs 8 to 29 disclosed in Table 3.

TABLE 3
Canine HC-B variants:
The following sequences correspond to AA 234 to 331 of Seq ID No: 2. The indication
of the position of the mutations is based on the AA position of Seq ID No: 2. Mutated
AA are indicated in bold letters.

| Seq | | | |
|---|---|---|---|
| ID | Designated | | |
| No: | herein as | Sequence | Mutations |
| 8 | HC-B_L235(A/Q/V) | M(A/Q/V)GGPSVFIFPPKPKDTLLIARTPEVTCVV | L235(A/Q/V) |
| | | VDLDPEDPEVQISWFVDGKQMQTAKTQPREEQ | |
| | | FNGTYRVVSVLPIGHQDWLKGKQFTCKVNNKA | |
| | | LPSP | |
| 9 | HC-B_L235A | MAGGPSVFIFPPKPKDTLLIARTPEVTCVVVDLD | L235A |
| | | PEDPEVQISWFVDGKQMQTAKTQPREEQFNGT | |
| | | YRVVSVLPIGHQDWLKGKQFTCKVNNKALPSP | |
| 10 | HC-B_S239(A/V) | MLGGP(A/V)VFIFPPKPKDTLLIARTPEVTCVVV | S239(A/V) |
| | | DLDPEDPEVQISWFVDGKQMQTAKTQPREEQF | |
| | | NGTYRVVSVLPIGHQDWLKGKQFTCKVNNKAL | |
| | | PSP | |
| 11 | HC-B_S239A | MLGGPAVFIFPPKPKDTLLIARTPEVTCVVVDLD | S239A |
| | | PEDPEVQISWFVDGKQMQTAKTQPREEQFNGT | |
| | | YRVVSVLPIGHQDWLKGKQFTCKVNNKALPSP | |
| 12 | HC-B_D270(A/N) | MLGGPSVFIFPPKPKDTLLIARTPEVTCVVVDLD | D270(A/N) |
| | | PE(A/N)PEVQISWFVDGKQMQTAKTQPREEQF | |
| | | NGTYRVVSVLPIGHQDWLKGKQFTCKVNNKAL | |
| | | PSP | |
| 13 | HC-B_D270A | MLGGPSVFIFPPKPKDTLLIARTPEVTCVVVDLD | D270A |
| | | PEAPEVQISWFVDGKQMQTAKTQPREEQFNGT | |
| | | YRVVSVLPIGHQDWLKGKQFTCKVNNKALPSP | |
| 14 | HC-B_P331(G/A/S) | MLGGPSVFIFPPKPKDTLLIARTPEVTCVVVDLD | P331(G/A/S) |
| | | PEDPEVQISWFVDGKQMQTAKTQPREEQFNGT | |
| | | YRVVSVLPIGHQDWLKGKQFTCKVNNKALPS | |
| | | (G/A/S) | |
| 15 | HC-B_P331G | MLGGPSVFIFPPKPKDTLLIARTPEVTCVVVDLD | P331G |
| | | PEDPEVQISWFVDGKQMQTAKTQPREEQFNGT | |
| | | YRVVSVLPIGHQDWLKGKQFTCKVNNKALPSG | |
| 16 | HC-B_L235 | M(A/Q/V)GGP(A/V)VFIFPPKPKDTLLIARTPEVT | L235(A/Q/V) |
| | (A/Q/V)_S239(A/V) | CVVVDLDPEDPEVQISWFVDGKQMQTAKTQPR | S239(A/V) |
| | | EEQFNGTYRVVSVLPIGHQDWLKGKQFTCKVN | |
| | | NKALPSP | |
| 17 | HC- | MAGGPAVFIFPPKPKDTLLIARTPEVTCVVVDLD | L235A |
| | B_L235A_S239A, | PEDPEVQISWFVDGKQMQTAKTQPREEQFNGT | S239A |
| | or HC-B_LS | YRVVSVLPIGHQDWLKGKQFTCKVNNKALPSP | |
| 18 | HC- | M(A/Q/V)GGPSVFIFPPKPKDTLLIARTPEVTCVV | L235(A/Q/V) |
| | B_L235(A/Q/V)_ | VDLDPEDPEVQISWFVDGKQMQTAKTQPREEQ | P331(G/A/S) |
| | P331(G/A/S) | FNGTYRVVSVLPIGHQDWLKGKQFTCKVNNKA | |
| | | LPS(G/A/S) | |
| 19 | HC- | MAGGPSVFIFPPKPKDTLLIARTPEVTCVVVDLD | L235A |
| | B_L235A_P331G, | PEDPEVQISWFVDGKQMQTAKTQPREEQFNGT | P331G |
| | or HC-B_LP | YRVVSVLPIGHQDWLKGKQFTCKVNNKALPSG | |
| 20 | HC- | MLGGPSVFIFPPKPKDTLLIARTPEVTCVVVDLD | D270(A/N) |
| | B_D270(A/N)_ | PE(A/N)PEVQISWFVDGKQMQTAKTQPREEQF | P331(G/A/S) |
| | P331(G/A/S) | NGTYRVVSVLPIGHQDWLKGKQFTCKVNNKAL | |
| | | PS(G/A/S) | |
| 21 | HC- | MLGGPSVFIFPPKPKDTLLIARTPEVTCVVVDLD | D270A |
| | B_D270A_P331G, | PEAPEVQISWFVDGKQMQTAKTQPREEQFNGT | P331G |
| | or HC-B DP | YRVVSVLPIGHQDWLKGKQFTCKVNNKALPSG | |
| 22 | HC-B HC- | M(A/Q/V)GGP(A/V)VFIFPPKPKDTLLIARTPEVT | L235(A/Q/V) |
| | B_L235(A/Q/V)_ | CVVVDLDPE(A/N)PEVQISWFVDGKQMQTAKT | S239(A/V) |
| | S239(A/V)_D270(A/N) | QPREEQFNGTYRWVSVLPIGHQDWLKGKQFTC | D270(A/N) |
| | | KVNNKALPSP | |
| 23 | HC- | MAGGPAVFIFPPKPKDTLLIARTPEVTCVVVDLD | L235A |
| | B_L235A_S239A_ | PEAPEVQISWFVDGKQMQTAKTQPREEQFNGT | S239A |
| | D270A, or HC- | YRVVSVLPIGHQDWLKGKQFTCKVNNKALPSP | D270A |
| | B_LSD | | |
| 24 | HC- | M(A/Q/V)GGP(A/V)VFIFPPKPKDTLLIARTPEVT | L235(A/Q/V) |
| | B_L235(A/Q/V)_ | CVVVDLDPEDPEVQISWFVDGKQMQTAKTQPR | S239(A/V) |
| | S239(A/V)_P331 | EEQFNGTYRVVSVLPIGHQDWLKGKQFTCKVN | P331(G/A/S) |
| | (G/A/S) | NKALPS(G/A/S) | |
| 25 | HC- | MAGGPAVFIFPPKPKDTLLIARTPEVTCVVVDLD | L235A |
| | B_L235A_S239A_ | PEDPEVQISWFVDGKQMQTAKTQPREEQFNGT | S239A |
| | P331G, or HC- | YRVVSVLPIGHQDWLKGKQFTCKVNNKALPSG | P331G |
| | B_LSP | | |
| 26 | HC-B HC- | M(A/Q/V)GGP(A/V)VFIFPPKPKDTLLIARTPEVT | L235(A/Q/V) |
| | B_L235(A/Q/V)_ | CVVVDLDPE(A/N)PEVQISWFVDGKQMQTAKT | S239(A/V) |
| | S239(A/V)_D270(A/N)_ | QPREEQFNGTYRWSVLPIGHQDWLKGKQFTC | D270(A/N) |
| | P331(G/A/S) | KVNNKALPS(G/A/S) | P331(G/A/S) |
| 27 | HC- | MAGGPAVFIFPPKPKDTLLIARTPEVTCVVVDLD | L235A |
| | B_L235A_S239A_ | PEAPEVQISWFVDGKQMQTAKTQPREEQFNGT | S239A |
| | D270A_P331G or | YRVVSVLPIGHQDWLKGKQFTCKVNNKALPSG | D270A |
| | HC-B LSDP | | P331G |
| 28 | HC- | (A/Q/V)(A/Q/V)GGPSVFIFPPKPKDTLLIARTPEV | M234(A/Q/V) |
| | B_M234(A/Q/V)_ | TCVVVDLDPEDPEVQISWFVDGKQMQTAKTQP | L235(A/Q/V) |
| | L235(A/Q/V) | REEQFNGTYRVVSVLPIGHQDWLKGKQFTCKV | |
| | | NNKALPSP | |
| 29 | HC- | AAGGPSVFIFPPKPKDTLLIARTPEVTCVVVDLD | M234A |
| | B_M234A_L235A | PEDPEVQISWFVDGKQMQTAKTQPREEQFNGT | L235A |
| | or HC-B_ML | YRVVSVLPIGHQDWLKGKQFTCKVNNKALPSP | |

Preferably, the polypeptide according to the present invention comprises a sequence selected from SEQ ID NO: 18, 19, 26, 27, or 29. Most preferably, the polypeptide comprises SEQ ID NO: 19 or 27.

The polypeptide according to the present may be a binding molecule. The term “binding molecule” according to the present invention reinforces to polypeptides comprising at least one domain specifically binding to a ligand, preferably a polypeptide, most preferably an epitope. Most preferably, at the least one domain specifically binding to a ligand is complementarity determining region (CDR) of an antibody or antibody fragment.

Accordingly, in a preferred embodiment of the invention, the polypeptide according to the invention may be an antibody, antibody fragment, or a polypeptide comprising an antibody fragment. Preferably the antibody, antibody fragment, or polypeptide comprising an antibody fragment binds to an epitope as disclosed below.

The term “antibody” as used herein refers to any form of antibody such as monoclonal antibodies, including full length monoclonal antibodies, polyclonal antibodies, multispecific antibodies, such as bispecific antibodies.

The term “antibody fragment” or “antigen binding fragment” as used herein refers to all fragments of antibodies exhibiting an antigen binding property, i.e. antibody fragments that retain the ability to bind specifically to the antigen that is bound by the corresponding full-length antibody. “Antibody fragments” thus comprise at least one, but preferably all, CDR regions of the full length antibody from which they were derived. Examples of antigen binding fragments or antibody fragments include, but are not limited to, Fab, Fab′, F(ab′)2, and Fv fragments; diabodies; linear antibodies; single-chain antibody molecules, e.g., sc-Fv; nanobodies and multispecific antibodies formed from antibody fragments.

The polypeptide according to the invention may be a canine or feline antibody. The term “canine antibody” or “feline antibody” relates to antibodies having a sequence of the Fc region (canine or feline sequence) which has at least 96%, preferably 98%, more preferably 99%, and most preferably 100% identity to a fully canine or feline antibody with the exception of the mutations according to the invention. The term “fully canine or feline antibody” refers to an antibody entirely comprising sequences originating from canine or feline genes. In some cases this may be canine or feline antibodies that have the gene sequence of an antibody derived from a canine or feline chromosome with the modifications outlined herein. A “canine antibody” or a “feline antibody” may also be recombinantly produced in cells of a different species, such as mouse, human or hybridoma cells. The antibody may also be derived from a synthetic or semisynthetic antibody sequence library. These sequences may comprise sequences encodes by canine or felines genes as well as artificial sequences, such as for example artificial CDRs. Thus, the canine of feline antibodies may comprise modifications, such as carbohydrate attachments, which are typically not found in antibodies produces in canine or feline cells.

The polypeptide according to the present invention may also be a caninized or felinized antibody. A “caninized antibody” or “felinized antibody” is a form of an antibody that contains sequences from both canine and non-canine (e.g., murine) antibodies, respectively sequences from feline and non-feline (e.g., murine) antibodies. Typically, a caninized antibody or felinized antibody will comprise at least one, and typically two or all, CDRs from a non-canine or non-feline organism and substantially canine or feline sequences outside of the CDRs.

The polypeptide according to the invention may also be a chimeric antibody. A “chimeric antibody” is an antibody having the variable domain from a first antibody and the constant domain from a second antibody, where the first and second antibodies are from different species. The chimeric antibody may for example comprise variable domain from an antibody derived from a rodent, for example a mouse or rat antibody, and a canine or feline constant domain.

The polypeptide according to the invention may also be a fusion protein. A fusion protein may be the canine immunoglobulin heavy chain constant domain may be fused whole or in part to the extracellular domain of a cytokine or chemokine receptor or other trans-membrane proteins.

The binding properties and immune effector functions of the Fc of different canine immunoglobuline isotypes were determined by Bergeron and colleagues (Bergeron L M, McCandless E E, Dunham S (2014) Comparative functional characterization of canine IgG subclasses. Vet Immunol Immunopathol. 2014; 157(1-2):31-41 as shown in Table 4.

TABLE 4
Binding and effector properties of canine IgG Isotypes

| Canine IgG Isotype: | HC-A | HC-B | HC-C | HC-D |
|---|---|---|---|---|
| Induction of cytolytic activity (ADCC) | −−/+ | +++ | +++ | −− |
| Complement binding (human C1q) | −−/+ | ++ | ++ | −−/+ |
| Fc gamma receptor I (FcγRI) | + | ++ | ++ | −−/+ |
| Fc gamma receptor IIb (FcγRIIb) | + | + | + | + |
| Fc gamma receptor III (FcγRIII) | −− | + | −−/+ | −− |
| Fc neonatal receptor I (FcRn) | ++ | ++ | −−/+ | ++ |
| Reactivity with Protein A | −−/+ | +++ | −− | −− |
| Reactivity with protein G | ++ | ++ | ++ | ++ |

Wherein ‘+++’ indicates very tight binding or high reactivity, ‘++’ indicates good binding, ‘+’ indicates that some binding was observed, ‘−−/+’ indicates little to no activation/binding, and ‘−−’ represents no binding (in the above Table 4).

Fc gamma receptor I receptor (FcγRI) is generally also referred to as CD64. Fc gamma receptor III (FcγRIII) is generally also referred to as (CD16).

In a main aspect, the polypeptide according to the invention exhibits a reduced binding affinity to C1 q and/or an Fc receptor relative to a polypeptide comprising the corresponding wild type Fc region. Preferably, the Fc receptor to which the binding is reduced is FcγRI, FcγRIII. Reduced binding in accordance with the present invention may be characterized by an increase of the KD of the polypeptide to the respective receptor by at least 3-fold, at least 5-fold, at least 10-fold, at least 20-fold, at least 100-fold, at least 1000-fold, at least 10000-fold, or at least 100000-fold.

The binding of the polypeptide according to the invention to C1q and/or an Fc receptor may be determined by in vitro binding assays well known in the art. For example, the binding to C1q and/or Fc, especially FcγRI receptors may be determined as disclosed in Example 1 or sections 2.5 and 2.6 of Bergeron et al. 2014 or WO 2015/091910, all of which are incorporated herein by reference. Accordingly, the assays for determining binding to C1q and/or Fc, especially FcγRI receptors, as disclosed in Example 1 or sections 2.5 and 2.6 of Bergeron et al. 2014 or WO 2015/091910 form part of the disclosure content of the present application.

Antibodies, especially in large scale commercial processes, are commonly isolated and purified via Protein A binding. However, HC-B which exhibits the strongest Protein A binding also exhibits a strong binding to C1q and Fc gamma receptor I receptor (FcγRI), resulting in a the activation of the immune effector functions of the complement system (CDC) and induction of cytolytic activity (ADCC) which are unacceptable for many indications treated with therapeutic antibodies as discussed above.

Surprisingly, as shown in FIG. 2, already polypeptides comprising SEQ IDs 9, 13, and 15 comprising a single substitution of L235, D270 or P331, exhibited a weak or significantly reduced binding to FcγRI or C1q. Notably binding to FcγRI was substantially absent for polypeptides comprising SEQ IDs 9 and binding to C1q was substantially absent for polypeptides comprising SEQ ID 15.

More surprisingly binding to FcγRI as well as to C1q was essentially completely absent for polypeptides comprising SEQ ID 19, comprising only the two substitution of L235 and P331.

In contrast to prior art mutations disclosed in EP 2 705 057 A1, which comprise a mutation at Kabat position 297 resulting in a deglycosylation of the antibody, the polypeptides according to the present invention achieve a silencing of the constant region of antibodies, especially of the highly active isotype HC-B, without deglycosylating the antibody. As deglycosylation may have a negative impact on the clearance of the antibodies from the circulation, the present invention sparingly provides an advantage over the prior art.

Furthermore, as shown in FIG. 4, the mutated Fc fragment of the HC-B isotype according to the present invention maintain their ability to bind to neonatal Fc receptor (FcRn). Binding to FcRn is generally known to increase half-time of IgG by reducing lysosomal degradation in endothelial cells and bone-marrow derived cells. Thus, maintaining FcRn binding in polypeptides having significantly reduced or absent binding to FcγRI as well as to C1q is highly advantageous for recombinant therapeutic antibodies.

Accordingly, the polypeptides of the present invention may be characterised by a binding to FcRn which is not impaired or not substantially impaired relative to the corresponding wild type polypeptide. The binding to FcγRI and/or C1q of said polypeptides is preferably significantly reduced or diminished. The binding to FcRn by a polypeptide according to the invention which is not substantially impaired may be a binding characterized by an increase in KD of the polypeptide to FcRn a by less than 2-fold, less than 3-fold, less than 5-fold, less than 10-fold, less than 25-fold, or less than 50-fold. Binding to FcRn may be determined as disclosed in Example 1 of the application.

The present invention advantageously provides polypeptides comprising at least a canine or feline Fc fragment from immunoglobulin subtype HC-B, comprising at least one substitution of an amino acid selected from at least one of amino acid position 235, 239, 270, and/or 331 relative to the wild type Fc region, which have a reduced binding to FcγRI receptor and/or C1q but have a binding to Protein A which is not substantially impaired relative to the corresponding wild type polypeptide.

As shown in section 1.3 of Example 1, the polypeptides of the present invention could be purified via binding to Protein A. Accordingly, the polypeptide of the present invention may be characterised by a binding to Protein A which is not substantially impaired relative to the corresponding wild type polypeptide. The binding to FcγRI and/or C1q of said polypeptide is preferably significantly reduced or diminished. The binding of a polypeptide according to the invention to Protein A may be a binding characterized by change in KD of the polypeptide to Protein A by less than 2-fold, less than 3-fold, less than 5-fold, or less than 10-fold when comparing the mutated polypeptide with a respective wild-type polypeptide.

In a preferred embodiment, the polypeptide is a glycosylated polypeptide exhibiting significantly reduced or absent binding to FcγRI receptor and/or C1q, when comparing the mutated polypeptide with a respective wild-type polypeptide, and which exhibits binding to neonatal Fc receptor (FcRn) and Protein A as described above. The binding to FcRn and Protein A may be a binding characterized by a change in KD of the mutated polypeptide according to the invention in comparison the wild type as described afore.

As evidenced by the significantly reduced binding to FcγRI receptor and/or C1q, the present invention provides polypeptides as described above that induce significantly reduced immune effector functions in comparison to a polypeptide comprising the corresponding wild type Fc region upon administration to a subject. As shown in FIG. 4, binding to FcγRI receptor and/or C1 q of the polypeptides according to the invention may be substantially absent. Thus, upon administration of the polypeptides to a subject, immune effector functions in comparison to a polypeptide comprising the corresponding wild type Fc region may be substantially absent.

The subject to which the polypeptides are administered may be a subject with an uncompromised immune system. Preferably, the subject to which the polypeptide is administered is a canine or feline subject, more preferably a canine or feline patient (or a canine or feline animal).

The domain specifically binding to a ligand as described above may be binding to an epitope derived from a protein selected from 17-IA, 4-1 BB, 4Dc, 6-keto-PGF1a, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM 10, ADAM 12, ADAM 15, ADAM17/TACE, ADAMS, ADAM9, ADAMTS, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Ax1, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/ZIP, CBL, CCI, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11 a, CD11 b, CD11 c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD50, CD52, CD54, CD55, CD56, CD61, CD64, CD66e, CD74, CD80, CD89, CD95, CD123, CD133, CD137, CD138, CD140a, CD146, CD147, CD148, CD152, CD154, CD163, CD164, CEACAM5, CEACAM6, CFTR, CGRP, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CX3CR1, CXCL, CXCL1, CXCL2, CXCL3, CXCL4, CXCL5, CXCL6, CXCL7, CXCL8, CXCL9, CXCL10, CX3CL1, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxinl, EpCAM, Ephrin B2/EphB4, EPO, ERCC, E-selectin, ET-1, Factor IIa, Factor VII, Factor VIIIc, Factor IX, Factor XI, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-3 ligand, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gp130, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her1 (Erb-B1, EGFR), Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MM), HIV gp120, HIV 1 MB gp120 V3 loop, HLA, HLA-DR, HM1 0.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-13R IL-15, IL-18, IL-18R, IL-22, IL-23, IL-31, IL-31R IL-33, IL-33R, interferon (INF)-alpha, INF-beta, INF-gamma, Inhibin, iNOS, Insulin A-chain, Insulin B-chain, integrin alpha2, integrin alpha3, integrin alpha4, integrin alpha4/beta1, integrin alpha4/beta7, integrin alpha5 (alphaV), integrin alpha5/beta1, integrin alpha5/beta3, integrin alpha6, integrin beta1, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bp1, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-14, MMP-15, MMP-2, MMP-24, MMP-3, MMP-7, MMP-8, MMP-9, MMP-13, MMP-3, MMP-1, MPIF, Mpo, MSK, MSP, mucin (Mud), MUC18, Muellerian-inhibitin substance, Mug, MuSK, Nav1.3, Nav1.5, Nav1.7, NAIP, NAP, NCAD, N-Cadherin, NCA 90, NCAM, NCAM, Neprilysin, Neurotrophin-3, -4, or -6, Neurturin, NGF, NGFR, NGF-beta, nNOS, NO, NOS, Npn, NRG-3, NT, NTN, OB, OGG1, OPG, OPN, OSM, OX40L, OX40R, p150, p95, PADPr, Parathyroid hormone, PARC, PARP, PBR, PBSF, PCAD, P-Cadherin, PCNA, PDGF, PD-1, PD-L1, PDK-1, PECAM, PEM, PF4, PGE, PGF, PGI2, PGJ2, PIN, PLA2, placental alkaline phosphatase (PLAP), PIGF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, 5100, SCF/KL, SDF-1, SERINE, Serum albumin, sFRP-3, Shh, SIGIRR, SK-1, SLAM, SLPI, SMAC, SMDF, SMOH, SOD, SPARC, ST2, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta Pan Specific, TGF-beta RI (ALK-5), TGF-beta RII, TGF-beta RIIb, TGF-beta Rill, TGF-beta 1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-beta, TNF-beta2, TNF-a, TNFR1, TNFc, TNF-RII, TNFRSF10A (TRAIL R1 Apo-2, DR4), TNFRSF10B (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11 B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF RI CD120a, p55-60), TNFRSF1 B (TNF RII CD120b, p75-80), TNFRSF26 (TNFRH3), TNFRSF3 (LTbR TNF RIM, TNFC R), TNFRSF4 (OX40ACT35, TXGP1R), TNFRSF5 (CD40 p50), TNFRSF6 (FasApo-1, APT1, CD95), TNFRSF6B (DcR3 M68, TR6), TNFRSF7 (CD27), TNFRSF8 (CD30), TNFRSF9 (4-1 BB CD137, I LA), TNFRSF21 (DR6), TNFRSF22 (DcTRAIL R2 TNFRH2), TNFRST23 (DcTRAIL R1 TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAKApo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-a Conectin, DIF, TNFSF2), TNFSF1 B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand, CD70), TNFSF8 (CD30 Ligand CD153), TNFSF9 (4-1 BB Ligand CD137 Ligand), TSLP, TSLPR, TARC, TP-1, t-PA, Tpo, TRAIL, TRAIL R, TRAIL-R1, TRAIL-R2, TRANCE, transferring receptor, TRF, Trk, TROP-2, TSG, tumor-associated antigen CA 125, tumor-associated antigen expressing Lewis Y related carbohydrate, TWEAK, TXB2, Ung, uPA, uPAR, uPAR-1, Urokinase, VCAM, VCAM-1, VECAD, VE-Cadherin, VE-cadherin-2, VEFGR-1 (flt-1), VEGF, VEGFR, VEGFR-3 (fit-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT1 OA, WNT1 OB, WNT11, WNT16, XCL1, XCL2, XCR1, XCR1, XEDAR, XIAP, XPD, and/or receptors for hormones and growth factors, toxins, parasite epitopes, bacterial epitopes and/or viral epitopes.

Preferably the epitope is derived from a protein selected from CTLA-4, EGF, Her1 (Erb-B1, EGFR), IgE, IL-1, IL-1R, IL-2, IL-2R, IL-4, IL-4R, IL-5, IL-5R, IL-6, IL-6R, IL-10, IL-12, IL-17, IL-17R, IL-18, IL-18R, IL-23, IL-31, IL-31R IL-33, IL-33R, integrin alpha4/beta7, NGF, TNF-alpha, PD-1. PD-L1, and/or VEGF.

An “epitope derived from” a molecule, especially a protein, may be a peptide epitope comprised within the sequence of the respective target or may be a conformational epitope established by the structure of the respective target.

In a further aspect, the invention relates to a pharmaceutical composition comprising the polypeptides as described herein, optionally together with a pharmaceutical acceptable carrier. A “pharmaceutical composition” is a composition comprising the polypeptide according to the invention and additional compounds which are toxicologically acceptable and enable the storage and the administration of the polypeptide according to the invention to a subject to be treated and allows the polypeptide to exert its intended pharmacological and biological activity.

The pharmaceutically acceptable carrier may include agents, e.g. diluents, stabilizers, adjuvants or other types of excipients that are non-toxic to the cell or mammal to be exposed thereto at the dosages and concentrations employed. Examples of pharmaceutically acceptable carriers include alumina; aluminum stearate; lecithin; serum proteins, such as human serum albumin, canine or other animal albumin; buffers such as phosphate, citrate, tromethamine or HEPES buffers; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, zinc salts, colloidal silica, or magnesium trisilicate; polyvinyl pyrrolidone, cellulose-based substances; polyethylene glycol; sucrose; mannitol; or amino acids including, but not limited to, arginine.

The invention further relates to the polypeptides or the pharmaceutical compositions described herein for use in a method of treating a disease. Likewise, the invention relates to the use the polypeptides or the pharmaceutical compositions described herein in a method of treating a disease. The method of treating a disease encompasses the step of administering the polypeptides or the pharmaceutical compositions described herein to a patient in need of treatment, preferably to a canine of feline subject.

In a preferred embodiment, the disease is an inflammatory disease, an allergy, a cancer, a pain, an (auto)-immune disease, a neurological disorder, an eye diseases, a cardiovascular dysfunctions or an infectious disease.

Preferably the inflammatory disease may be selected from rheumatoid arthritis, osteoarthritis, psoriasis, atopic dermatitis, and inflammatory bowel disease; the allergy may be asthma, the cancer may be selected from lymphoma, melanoma, hemangiosarcoma, mast cell tumors, osteosarcoma, brain cancers, breast cancer, bowel cancer); the pain may be selected from osteoarthritic pain, cancer pain, lower back pain, post-operative pain, neuropathic or inflammatory pain; the (auto)-immune diseases may be selected from systemic lupus erythematosus; the neurological disorders may be selected from epilepsy; the eye diseases may be selected from age related macular degeneration; the cardiovascular dysfunctions may be selected from hypertension, congestive heart failure; and the infectious disease may be selected from hepatitis, distemper, canine infectious respiratory disease, feline immunodeficiency virus.

The Fc domain, or the antibody, or the Fc-fusion protein of the invention may be for use in the treatment of infectious or parasitic diseases, which may be selected among diseases induced by ectoparasites and endoparasites of dogs, and respiratory infections, urinary infections and dermatological infections, notably skin infections, soft tissues infections and otitis.

As mentioned above, the antibody and the Fc-fusion protein of the invention may be used for therapeutic, diagnostic or for research uses or methods.

In a further aspect, the invention relates to a polynucleotide encoding the polypeptide according to the invention. The polynucleotide may be an isolated polynucleotide. The polynucleotide may be comprised in a vector, such as a plasmid or an artificial chromosome. The polynucleotide may be operatively linked to transcriptional and translational control sequences. In this context, the term “operatively linked” means that a transcriptional and translational control sequences serve to functionally transcribe and translate the polynucleotide to express the encoded polypeptide.

The vector may be comprised in a cell. The cell is preferably a host cell suitable for recombinantly expressing antibodies or antibody fragments. Exemplary eukaryotic cells include mammalian cells, such as primate or non-primate animal cells; yeast cells; plant cells; and insect cells. Non-limiting exemplary mammalian cells include, but are not limited to, NSO cells, 293 cells, and CHO cells, and cell lines derived therefrom, for example 293-6E, DG44, CHO-S, and CHO-K cells.

In a further aspect, the present invention relates to a method of generating a polypeptide comprising an Fc fragment, wherein the method comprises at least the steps of:
introducing at least one mutation into a polynucleotide encoding at least one Fc fragment, wherein the mutation in the polynucleotide results in a substitution of at least one amino acid as disclosed above within the of the polypeptide encoded by the polynucleotide;
expressing the polypeptide comprising a Fc fragment comprising said at least one substitution in a host cell.

Preferably, the mutated polynucleotide encodes at least one of SEQ ID NOs: 1 to 29 as described above.

Furthermore, the invention relates to a method of reducing the immune effector function of a polypeptide comprising an Fc fragment, wherein the method comprises the steps of:
introducing at least one mutation into polypeptide comprising a Fc fragment, wherein the mutation in the polynucleotide results in a substitution of at least one amino acid as disclosed above in said polypeptide comprising an Fc fragment encoded by the polynucleotide;
expressing the polypeptide comprising an Fc fragment comprising said at least one substitution in a host cell.

The present invention shall be explained in more detail by the following figures and examples.

### FIGURES

FIG. 1 shows a schematic overview of variants tested in this study.

FIG. 2 shows the binding of wild type as well as mutant variants to either C1q (A) or FcγRI (B). All IgG variants were produced as cell culture supernatants (ccSups) and tested at different dilutions. IgGs derived from the supernatants were captured by binding to the target antigen for detection of binding to C1q or FcγRI. Signals are depicted as bars and are normalized to the HC-B wild type variant showing maximal binding to C1q or FcγRI.

FIG. 3 shows the binding of wildtype as well as mutant variants to either C1q (A) or FcγRI (B and C). All IgG variants were produced as cell culture supernatants (ccSups) and tested at different dilutions. While IgGs derived from the supernatants were captured by binding to the target antigen for detection of binding to C1q (A), IgGs were captured via antigen (B) as well as by Fab anti-canine IgG (C) for detection of binding to FcγRI. Signals are depicted as bars and are normalized to the HC-B wildtype variant showing maximal binding to C1q or FcRI. Combining two or more mutations yield Fc-variants with completely abolished binding to either C1q or FcRI.

FIG. 4 shows the binding of selected variants to C1q (A), FcγRI (B) and FcRn (C). All IgG variants were tested as purified IgG at different concentrations. Only the wildtype HC-B variant shows binding on C1q and FcγRI, whereas other isoforms even at high concentration do not bind the respective molecules. Comparable binding to the FcRn receptor was observed for HC-B-LP and HC-B_LSDP, interestingly HC-A wt shows only minimal binding. Mutations in the Fc-part of the antibody variants has no impact on antigen recognition (D).

FIG. 5 shows the binding of selected variants selected variants to C1q (A), FcγRI (B) and FcRn (C). All IgG variants were tested as purified IgG at different concentrations. Wildtype HC-B variant shows strong binding on C1q and FcγRI, whereas HC-A wt even at high concentration does not bind the respective molecules. The Fc-engineered HC-B variant HC-B_ML shows slightly reduced binding to C1q but completely lacks FcγRI binding. Comparable binding to the FcRn receptor was observed for HC-B wt and HC-B_ML, interestingly HC-A wt shows only minimal binding. Mutations in the Fc-part of the antibody variants has no impact on antigen recognition (D).

FIG. 6 shows a comparison of wild type sequences of constant regions of canine IgG isotype HC-A (SEQ ID NO: 1), canine IgG isotype HC-B (SEQ ID NO: 2), canine IgG isotype HC-C (SEQ ID NO: 3), canine IgG isotype HC-D (SEQ ID NO: 4), feline IgG 1a (SEQ ID NO: 5), feline IgG 1b (SEQ ID NO: 6), and feline IgG 2 (SEQ ID NO: 7) with the human IgG1 constant region.

### NUMBERED ASPECTS AND EMBODIMENTS OF THE INVENTION

1. A polypeptide comprising at least a canine or feline Fc fragment, wherein the Fc fragment comprises at least one substitution of an amino acid selected from at least one of amino acid position 235, 239, 270, and/or 331, relative to the wild type Fc fragment, preferably selected from at least one of L235, S239, D270, and/or P331.
2. The polypeptide according to aspect 1, wherein the wild type sequence (the sequence of the wild type Fc fragment) is selected from any of Seq ID NOs: 1 to 7, the sequences (according to Kabat numbering) of GeneBank accession Nos. AF354264, AF354265, AF354266, AF354267, in particular the sequences of amino acids 234 to 331 (according to Kabat numbering) of GeneBank accession Nos. AF354264, AF354265, AF354266, AF354267, or the wild type sequences as disclosed by Striezel et al., page 220.
3. The polypeptide according to aspect 1 or 2, wherein the Fc fragment comprises at least two substitutions of amino acids selected from at least two of amino acid positions 234, 235, 239, 270, and/or 331, preferably from L235 and S239, L235 and D270, L235 and P331, L235 and P331, S239 and D270, S239 and P331, D270 and P331, M234 and L235, M234 and S239; M234 and D270; M234 and P331.
4. The polypeptide according to any of the preceding aspects, wherein the Fc fragment comprises at least two substitutions of amino acids selected from amino acid positions 234, 235, 239, 270, and/or 331 wherein at least one of the two amino acid positions is selected from 239, 270, and/or 331.
5. The polypeptide according to any of the preceding aspects, wherein the Fc fragment comprises at least three substitutions of amino acids selected from at least three of amino acid position 234, 235, 239, 270, and/or 331, preferably L235, S239, and D270; S239, D270, and P331; L235, D270, and P331; or L235, S239, and P331.
6. The polypeptide according to any of the preceding aspects, wherein the Fc fragment comprises at least four substitutions selected from amino acid positions 234, 235, 239, 270, and 331, preferably of amino acids 235, 239, 270, and 331.
7. The polypeptide according to any of the preceding aspects, wherein the one or more substitution is a substitution by alanine, or glycine, preferably wherein the one or more substitution is L235A, S239A, D270A, and/or P331G.
8. The polypeptide according to any of the preceding aspects, wherein the canine Fc fragment is an Fc fragment from IgG isotype IgG-A, IgG-B, IgG-C, or IgG-D, most preferably from IgG isotype IgG-B.
9. The polypeptide according any of the preceding aspects comprising a sequence selected from SEQ ID NOs 8 to 29, preferably SEQ ID NO 18, 19, 26, 27, or 29.
10. The polypeptide according any of the preceding aspects, wherein the polypeptide is a binding molecule, preferably an antibody, antibody fragment, or a polypeptide comprising an antibody fragment.
11. The polypeptide according to any of the preceding aspects, wherein the polypeptide has a reduced binding affinity to C1q and/or an Fc receptor relative to a polypeptide comprising the corresponding wild type Fc fragment.
12. The polypeptide according to aspect 11, wherein the Fc receptor is an FcγRI, or FcγRIII.
13. The polypeptide according to any of the preceding aspects, characterised by a binding to FcRn and/or Protein A which is not substantially impaired relative to the corresponding wild type polypeptide.
14. The polypeptide according to any of the preceding aspects, wherein the polypeptide induces reduced immune effector functions relative to a polypeptide comprising the corresponding wild type Fc fragment upon administration to a subject, wherein preferably the subject has an uncompromised immune system.
15. The polypeptide according to any of the preceding aspects, wherein the polypeptide induces reduced ADCC or CDC relative to a polypeptide comprising the corresponding wild type Fc fragment upon administration to a subject.
16. A polypeptide according to any of the preceding aspects, wherein the polypeptide comprises a domain specifically binding to an epitope derived from a protein selected from CTLA-4, EGF, Her1 (Erb-B1, EGFR), IgE, IL-1, IL-1R, IL-2, IL-2R, IL-4, IL-4R, IL-5, IL-5R, IL-6, IL-6R, IL-10, IL-12, IL-17, IL-17R, IL-18, IL-18R, IL-23, IL-31, IL-31R IL-33, IL-33R, integrin alpha4/beta7, NGF, TNF-alpha, PD-1. PD-L1, VEGF.
17. A pharmaceutical composition comprising a polypeptide according to any of the preceding aspects.
18. The polypeptide or composition according to any of the preceding aspects for use in a method of treating a disease in a canine or feline subject, preferably wherein the disease is an inflammatory disease, an allergy, a cancer, a pain, an (auto)-immune disease, a neurological disorder, an eye diseases, a cardiovascular dysfunctions or an infectious disease.
19. A polynucleotide encoding the polypeptide according to the preceding aspect.
20. A vector or cell comprising the polynucleotide according aspect 19.
21. A method of generating a polypeptide comprising an Fc C2 and C3 fragment, wherein the method comprises at least the steps of:
   introducing at least one mutation into a polynucleotide encoding at least one Fc C2 and C3 fragment,
wherein the mutation in the polynucleotide results in a substitution of at least one amino acid as disclosed above within the of the polypeptide encoded by the polynucleotide;
   expressing the polypeptide comprising a Fc C2 and C3 fragment comprising said at least one substitution in a host cell.
22. A method of reducing the immune effector function of a polypeptide comprising an Fc C2 and C3 fragment, wherein the method comprises the steps of:
   introducing at least one mutation into polypeptide comprising a Fc C2 and C3 fragment, wherein the mutation in the polynucleotide results in a substitution of at least one amino acid as disclosed above in said polypeptide comprising an Fc C2 and C3 fragment encoded by the polynucleotide;
   expressing the polypeptide comprising an Fc C2 and C3 fragment comprising said at least one substitution in a host cell.
23. A method of treating a disease in a canine or feline subject, comprising administering (a therapeutically effective amount) of the polypeptide or (pharmaceutical) composition according to any of the preceding aspects to a canine or feline subject in need thereof, preferably wherein the disease is an inflammatory disease, an allergy, a cancer, a pain, an (auto)-immune disease, a neurological disorder, an eye diseases, a cardiovascular dysfunctions or an infectious disease.

### EXAMPLES

### Example 1

1. Material and Methods:

1.1 Construction of Variants

A fully canine anti-GFP antibody was used as a model antibody to study C1q and FcγRI interaction of Fc-mutants containing different constant antibody regions. The antibody was derived from a fully canine phage display library as described in WO 2018/234438.

In total, 13 constructs were generated including constructs comprising altered Fc fragments in accordance with SEQ ID NOs: 9, 11, 13, 15, 17, 19, 21, 23, 25, 27, and 29 have been, thus four HC-B variants with mutation of one amino acid and six HC-B variants with combinations of these single mutations, as well as wildtype HC-B as positive control and wildtype HC-A described to lack effector function as a negative control in binding experiments.

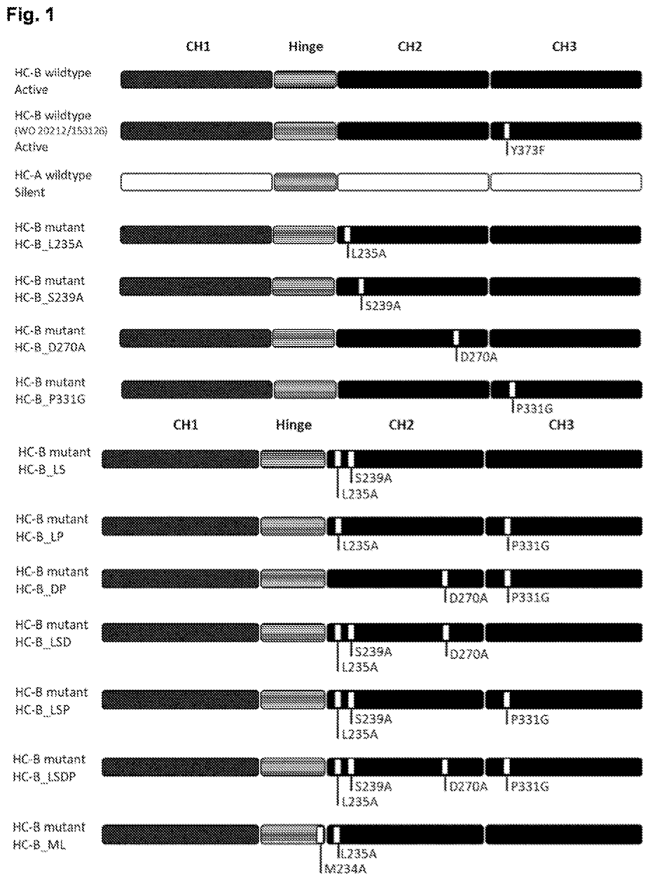

The constructs are schematically depicted in FIG. 1.

Mutant Fc constructs were synthesized by PCR mutagenesis and cloned into a proprietary mammalian expression vector encoding both heavy and light chain sequences of the anti-GFP IgG antibody.

1.2 Production of IgG-Containing Cell Culture Supernatants for Screening

HEK293-EBNA cells were transfected with mammalian expression vector DNA using jetPRIME® transfection reagent (Polyplus-transfection®). Cell culture supernatants were harvested on day 3 post transfection and concentrations of IgGs were determined by ELISA (data not shown). Supernatants were used for binding assays.

1.3 Production and Purification of IgGs

HEK293F suspension cells were grown in log phase and transfected with mammalian expression vector DNA using FectoPRO (Polyplus-transfection®). Cell culture supernatants were harvested on day 8 post transfection and subjected to standard Protein A affinity chromatography (MabSelect SURE, GE Healthcare). Buffer exchange was performed to 1× Dulbcecco's PBS and (pH 7.2) and samples were sterile filtered (0.2 μm pore size). Protein concentrations were determined by UV-spectrophotometry and purities of IgG were analyzed under denaturing, reducing using SDS-PAGE and by size exclusion chromatography (SEC). SEC was performed on an ÄKTA Purifier system (GE Healthcare Europe GmbH, Freiburg, Germany). For separation a Superdex75 HR 10/30 column was used 30 (GE Healthcare Europe GmbH, Freiburg, Germany). For each sample 10 μl of protein was loaded onto the column, separation was performed at a flow rate of 0.05 ml/min and recorded analyzing the UV absorption at 260 and 280 nm. The running buffer was composed of Gibco D-PBS, pH 7.4 (Invitrogen, Paisley, USA).

Quality control following transfection and purification reveals that antibody variants used in binding experiments are highly pure and monomeric which rules out the possibility that variances in C1q or FcγRI binding are due to the presence of e.g. aggregates within the protein preparation (data not shown). Interestingly, also the HC-A type antibody could be readily purified using Protein A demonstrated by the lack of detectable antibody in the flow through or wash suggesting all protein was captured on Protein A (data not shown).

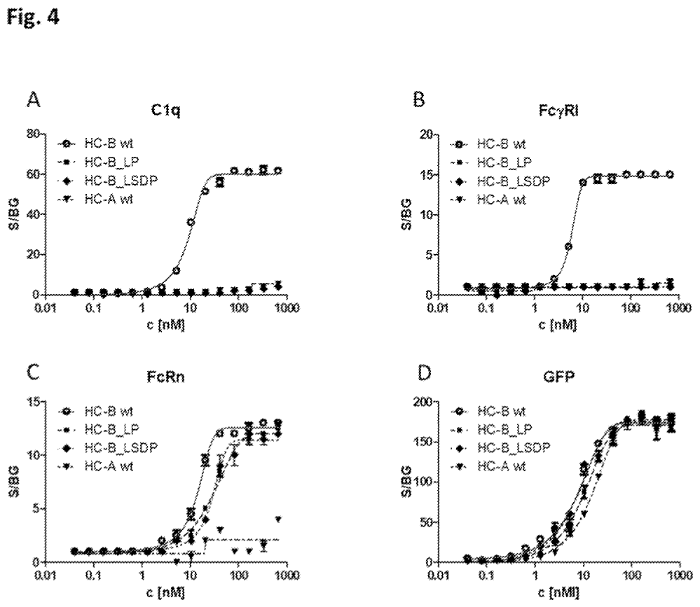

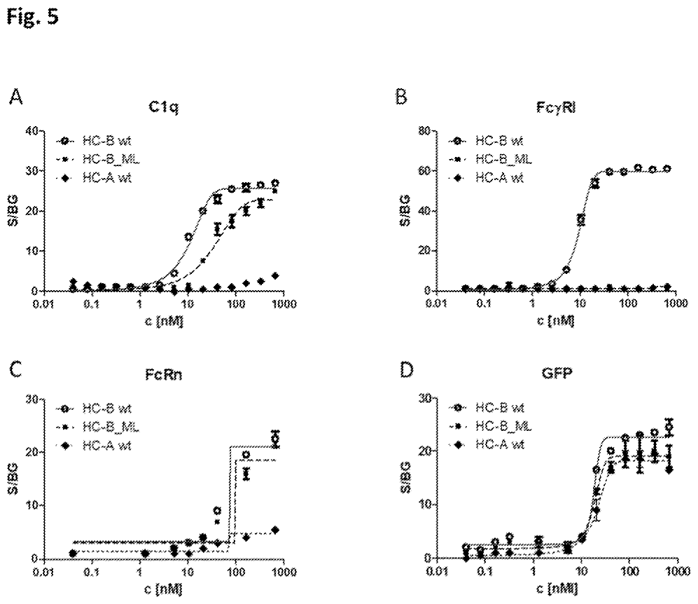

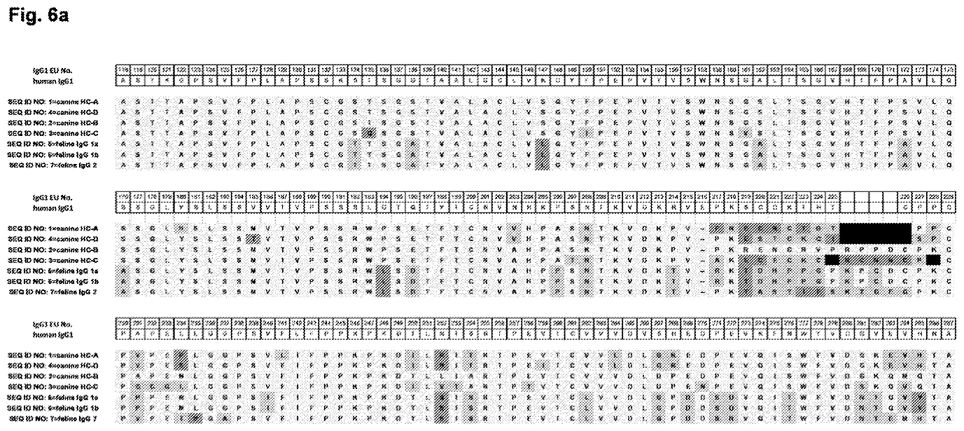

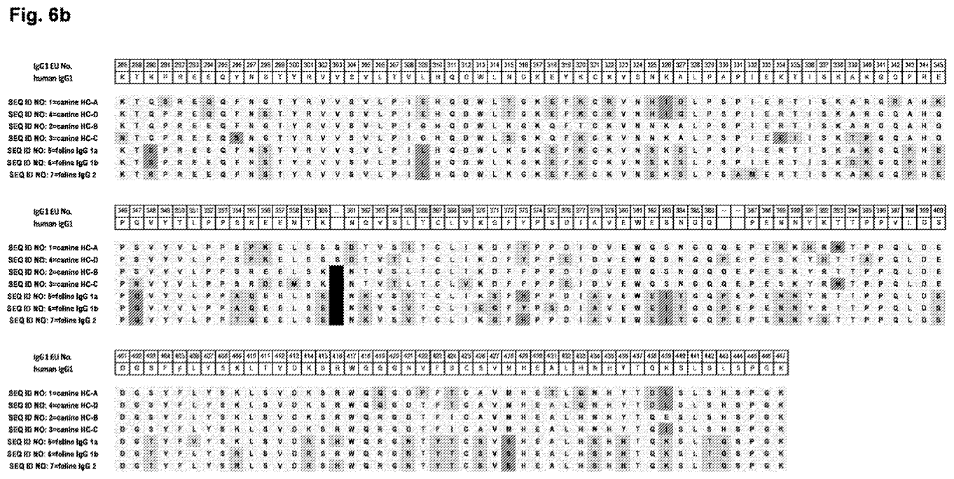

Also, all antibody constructs had the same binding efficiency to their target protein GFP (FIG. 4 and FIG. 5).

1.4 C1q & FcγRI ELISA Using Cell Culture Supernatants

Antibody binding to complement protein C1q was assessed by ELISA. Briefly, Maxisorp plates (Nunc™) were coated with GFP (3 μg/ml) for 1 h at room temperature (RT). Plates were blocked using 5% skimmed milk in PBS. Antibody containing supernatants were titrated in PBS and incubated on immobilized GFP for 1 h at room temperature, shaking. Recombinant purified human C1q protein (Quidel Corporation, San Diego, CA, USA) was added to bound antibodies at a concentration of 10 μg/ml in M-PBST (PBS supplemented with 0.5% skimmed milk and 0.05% Tween-20) and plates were incubated for 1 h at room temperature gently shaking. Following washing steps with PBS-T (PBS supplemented with 0.05% Tween-20) binding was detected using a sheep-anti-human C1q antibody coupled to HRP (Bio-Rad). Plates were developed using the QuantaBlu fluorogenic peroxidase substrate kit (Thermo) according to the manufacturer's instructions and fluorescence was measured at a Genios Reader Pro (Tecan) using excitation at 320 nm and emission at 430 nm.

Antibody binding to human FcγRI was assessed by ELISA. Briefly, Maxisorp plates (Nunc™) were coated with either GFP (3 μg/ml) or Fab anti-canine IgG (H+L) (5 μg/ml) for 1 h at room temperature (RT). Plates were blocked using ChemiBLOCKER (Millipore). Antibody containing supernatants were titrated in PBS and incubated on immobilized GFP for 1 h at room temperature, shaking. Recombinant biotinylated human FcγRI protein (Sino Biological) was added to bound antibodies at a concentration of 1 μg/ml in PBS supplemented with 10% ChemiBLOCKER and 0.05% Tween-20 and plates were incubated for 1 h at room temperature gently shaking. Following washing steps with PBS-T (PBS supplemented with 0.05% Tween-20) binding was detected using Streptavidin-HRP (Jackson ImmunoResearch). Plates were developed using the QuantaBlu fluorogenic peroxidase substrate kit (Thermo) according to the manufacturer's instructions and fluorescence was measured at a Genios Reader Pro (Tecan) using excitation at 320 nm and emission at 430 nm.

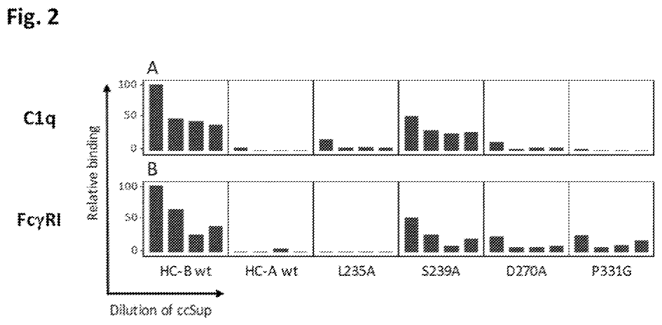

As shown in FIG. 2, the HC-B wildtype variant bound well to both C1q (1A, graph on the left hand side) or FcγRI protein (1B, graph on the left hand side). Also, as expected, the wildtype HC-A antibody did not or very weakly bind to C1q or FcγRI. All single mutations exhibit lower binding to either C1q or FcγRI compared to wildtype HC-B. However, there are clear differences between the individual variants. Whereas the L235A mutation significantly reduced binding to C1q and diminished FcγRI binding, P331G to some extent retained binding to FcγRI but does not recognize C1q. The S239A and D270 variant showed reduced binding to both proteins, however, the effect is more dominant for D270A.

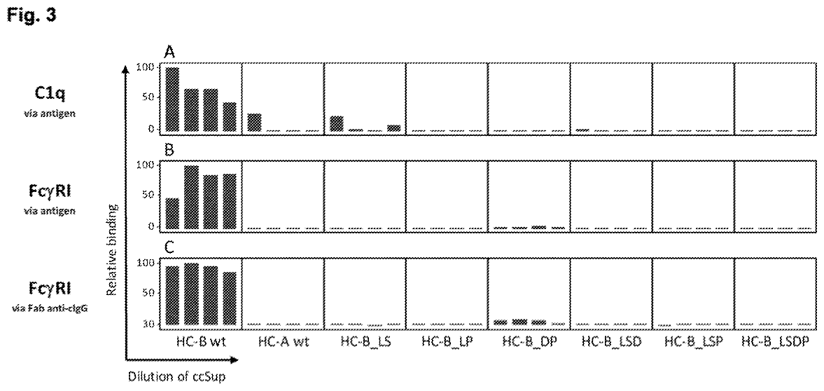

As none of the single mutants showed complete lack of binding to both C1q and FcγRI, different combinations were tested. Results are shown in FIG. 3, indicating that a combination of two or more mutations can abolish binding to C1q and FcγRI. With the exception of variant HC-B_LS and HC-B_DP that show little residual binding on either C1q or FcγRI, all other variants did not bind under the experimental settings.

To verify results from the screening assay several variants were purified and tested for binding to C1q, FcγRI as well as FcRn in a concentration dependent manner in comparison to HC-B wt and HC-A wt.

1.5 Characterization of Fc-Variants Using Purified Antibodies

1.5.1 C1q ELISA with Purified IgGs

Binding of purified IgGs to complement protein C1q was assessed by ELISA essentially as described above. Briefly, purified antibodies were titrated in PBS and immobilized onto Maxisorp plates (Nunc™) for 1 h at room temperature. Plates were blocked using 5% skimmed milk in PBS. Recombinant purified human C1q protein (Quidel Corporation, San Diego, CA, USA) was added to bound antibodies at a concentration of 10 μg/ml in M-PBST (PBS supplemented with 0.5% skimmed milk and 0.05% Tween-20) and plates were incubated for 1 h at room temperature gently shaking. Following washing steps with PBS-T (PBS supplemented with 0.05% Tween-20) binding was detected using a sheep-anti-human C1q antibody coupled to HRP (Bio-Rad). Plates were developed using the QuantaBlu fluorogenic peroxidase substrate kit (Thermo) according to the manufacturer's instructions and fluorescence was measured at a Genios Reader Pro (Tecan) using excitation at 320 nm and emission at 430 nm.

1.5.2 FcγRI ELISA with Purified IgGs

Antibody binding to human FcγRI was assessed by ELISA. Briefly, antibodies were titrated in PBS and immobilized onto Maxisorp plates (Nunc) for 1 h at room temperature. Plates were blocked using ChemiBLOCKER. Recombinant biotinylated human FcγRI protein (Sino Biological) was added to bound antibodies at a concentration of 1 μg/ml in PBS supplemented with 10% ChemiBLOCKER and 0.05% Tween-20 and plates were incubated for 1 h at room temperature gently shaking. Following washing steps with PBS-T (PBS supplemented with 0.05% Tween-20) binding was detected using Streptavidin-HRP (Jackson ImmunoResearch). Plates were developed using the QuantaBlu fluorogenic peroxidase substrate kit (Thermo) according to the manufacturer's instructions and fluorescence was measured at a Genios Reader Pro (Tecan) using excitation at 320 nm and emission at 430 nm.

1.5.3 FcRn-ELISA with Purified IgGs

Antibody binding to canine FcRn was assessed by ELISA. Briefly, antibodies were titrated in PBS and immobilized onto Maxisorp plates (Nunc) for 1 h at room temperature. Plates were blocked using ChemiBLOCKER. Recombinant biotinylated canine FcRn protein (Immunitrack) was added to bound antibodies at a concentration of 10 μg/ml in PBS supplemented with 10% ChemiBLOCKER and 0.05% Tween-20 at pH 6 and plates were incubated for 1 h at room temperature gently shaking. Following washing steps with PBS-T (PBS supplemented with 0.05% Tween-20) binding was detected using Streptavidin-HRP (Jackson ImmunoResearch). Plates were developed using the QuantaBlu fluorogenic peroxidase substrate kit (Thermo) according to the manufacturer's instructions and fluorescence was measured at a Genios Reader Pro (Tecan) using excitation at 320 nm and emission at 430 nm.

1.5.4 Antigen-Binding ELISA

Antibody binding to model antigen GFP was assessed by ELISA. GFP at 3 μg/mL diluted in PBS was immobilized onto Maxisorp plates (Nunc) for 1 h at room temperature. Plates were blocked using 5% skimmed milk in PBS. Antibodies were titrated in M-PBST (PBS supplemented with 0.5% skimmed milk and 0.05% Tween-20), added to the bound antigen and plates were incubated for 1 h at room temperature gently shaking. Following washing steps with PBS-T (PBS supplemented with 0.05% Tween-20) binding was detected using rabbit-anti-canine (Fab)₂ antibody coupled to HRP (Sigma). Plates were developed using the QuantaBlu fluorogenic peroxidase substrate kit (Thermo) according to the manufacturer's instructions and fluorescence was measured at a Genios Reader Pro (Tecan) using excitation at 320 nm and emission at 430 nm.

2. Results

HC-B wt is known to efficiently induce effector functions which is mediated via binding to the proteins C1q and FcγRI and is used as a control in the subsequent experiments as a positive control. In vitro binding experiments from previous studies revealed that canine HC-A binds C1q protein and FcγRI with little to no affinity which results in a lack of effector function. Selected candidates were purified and tested against wildtype HC-B and HC-A. Variant HC-B_ML contains the double mutation M234A/L235A which for human antibodies has been described to significantly reduce effector function (Xu D, Alegre M L, Varga S S, et al. In vitro characterization of five humanized OKT3 effector function variant antibodies. Cell Immunol. 2000; 200(1):16-26.). Quite surprisingly, the same mutations in a canine framework abolished FcγRI binding but the variant was still able to bind C1q (see FIGS. 4 A and B). These results show that properties of mutations in the human Fc region are not generally transferable to the canine Fc region. Binding to the neonatal receptor FcRn (FIG. 4 C) and to the antigen the antibody was directed against (FIG. 4 D) were comparable. The HC-A wildtype gave the expected results but it was surprising to see that binding to FcRn was also reduced compared to the HC-B variants.

As also seen in the experiments using cell culture supernatants, the variants HC-B_LP and HC-B_LSDP were confirmed to have lost binding to C1q and FcγRI even at high antibody concentrations (FIGS. 5 A and B) whereas binding to FcRn could be retained and also recognition of the target antigen was unaffected.

1. A polypeptide comprising at least a canine or feline Fc fragment, wherein the Fc fragment comprises at least one substitution of an amino acid selected from at least one of amino acid position 235, 239, 270, and 331, relative to the wild type Fc fragment.

2. The polypeptide according to claim 1, wherein the wild type sequence is selected from any of Seq ID NOs: 1 to 7, the sequences of amino acids 234 to 331 (according to Kabat numbering) of GeneBank accession Nos. AF354264, AF354265, AF354266, AF354267 and disclosed by Striezel et al., page 220.

3. The polypeptide according to claim 1, wherein the Fc fragment comprises at least two substitutions of amino acids selected from at least two of amino acid positions 234, 235, 239, 270, and 331.

4. The polypeptide according to claim 1, wherein the Fc fragment comprises at least two substitutions of amino acids selected from amino acid positions 234, 235, 239, 270, and 331
wherein at least one of the two amino acid positions is selected from 239, 270, and 331.

5. The polypeptide according to claim 1, wherein the Fc fragment comprises at least three substitutions of amino acids selected from at least three of amino acid position 234, 235, 239, 270, and 331.

6. The polypeptide according to claim 1, wherein the Fc fragment comprises at least four substitutions selected from amino acid positions 234, 235, 239, 270, and 331.

7. The polypeptide according to claim 1, wherein the one or more substitution is a substitution by alanine, or glycine.

8. The polypeptide according to claim 1, wherein the canine Fc fragment is an Fc fragment from IgG isotype IgG-A, IgG-B, IgG-C, or IgG-D.

9. The polypeptide according to claim 1, wherein said polypeptide comprises a sequence selected from SEQ ID NOs 8 to 29.

10. The polypeptide according to claim 1, wherein the polypeptide is a binding molecule.

11. The polypeptide according to claim 1, wherein the polypeptide has a reduced binding affinity to C1q and/or an Fc receptor relative to a polypeptide comprising the corresponding wild type Fc fragment.

12. The polypeptide according to claim 11, wherein the Fc receptor is an FcγRI, or FcγRIII.

13. The polypeptide according to, claim 1, wherein said polypeptide's ability to bind to FcRn and/or Protein A is not substantially impaired relative to the corresponding wild type polypeptide.

14. The polypeptide according to claim 1, wherein the polypeptide induces reduced immune effector functions relative to a polypeptide comprising the corresponding wild type Fc fragment upon administration to a subject.

15. The polypeptide according to claim 1, wherein the polypeptide induces reduced ADCC or CDC relative to a polypeptide comprising the corresponding wild type Fc fragment upon administration to a subject.

16. A polypeptide according to claim 1, wherein the polypeptide comprises a domain specifically binding to an epitope derived from a protein selected from CTLA-4, EGF, Her1 (Erb-B1, EGFR), IgE, IL-1, IL-1R, IL-2, IL-2R, IL-4, IL-4R, IL-5, IL-5R, IL-6, IL-6R, IL-10, IL-12, IL-17, IL-17R, IL-18, IL-18R, IL-23, IL-31, IL-31R IL-33, IL-33R, integrin alpha4/beta7, NGF, TNF-alpha, PD-1, PD-L1 and VEGF.

17. A method of treating a disease in a canine or feline subject, wherein said method comprises administering to said subject a polypeptide comprising at least a canine or feline Fc fragment, wherein the Fc fragment comprises at least one substitution of an amino acid selected from at least one of amino acid position 235, 239, 270, and 331, relative to the wild type Fc fragment.

18. The polypeptide of claim 3, wherein the Fc fragment comprises at least two substitutions of amino acids selected from L235 and S239, L235 and D270, L235 and P331, L235 and P331, S239 and D270, S239 and P331, D270 and P331, M234 and L235, M234 and S239; M234 and D270; M234 and P331.

19. The polypeptide of claim 5, wherein the Fc fragment comprises at least three substitutions of amino acids selected from L235, S239, and D270: S239, D270, and P331; L235, D270, and P331; or L235, S239, and P331.

20. The polypeptide of claim 6, wherein the Fc fragment comprises at least four substitutions selected from amino acid positions 235, 239, 270, and 331.


SEQUENCE LISTING (29 sequences)

SEQ ID NO: 1 - protein, 330 aa, Canis lupus
  HC-A WT
STTAPSVFPLAPSCGSTSGSTVALACLVSGYFPEPVTVSWNSGSLTSGVHTFPSVLQSSGLHSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 2 - protein, 335 aa, Canis lupus
  HC-B WT
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 3 - protein, 333 aa, Canis lupus
  HC-C WT
ASTTAPSVFPLAPSCGSQSGSTVALACLVSGYIPEPVTVSWNSVSLTSGVHTFPSVLQSSGLYSLSSMVTVPSSRWPSETFTCNVAHPATNTKVDKPVAKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQISLSHSPGK

SEQ ID NO: 4 - protein, 331 aa, Canis lupus
  HC-D WT
ASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFYPPEIDVEWQSNGQPEPESKYHTTAPQLDEDGSYFLYSKLSVDKSRWQQGDTFTCAVMHEALQNHYTDLSLSHSPGK

SEQ ID NO: 5 - protein, 335 aa, Felis catus
  IgG1a
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSENKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 6 - protein, 335 aa, Felis catus
  IgG1b
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKDKGQPHEPQVYVLPPAQEELSENKVSVTCLIEGFYPSDIAVEWEITGQPEPENNYRTTPPQLDSDGTYFLYSRLSVDRSRWQRGNTYTCSVSHEALHSHHTQKSLTQSPGK

SEQ ID NO: 7 - protein, 334 aa, Felis catus
  IgG2
ASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVPKTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 8 - protein, 98 aa, Artificial Sequence
  HC-B_L235(A/Q/V)
  VARIANT 2: X = Ala, Gln, Val
MXGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSP

SEQ ID NO: 9 - protein, 98 aa, Artificial Sequence
  HC-B_L235A
MAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSP

SEQ ID NO: 10 - protein, 98 aa, Artificial Sequence
  HC-B_S239(A/V)
  VARIANT 6: X = Ala, Val
MLGGPXVFIFPPKPKDTLLIARTPEVTCVVVDLDPEDPEVQISWFVDGKQMQTAKTQPREEQFNGTYRVVSVLPIGHQDWLKGKQFTCKVNNKALPSP

SEQ ID NO: 11 - protein, 98 aa, Artificial Sequence
  HC-B_S239A
MLGGPAVFIFPPKPKDTLLIARTPEVTCVVVDLDPEDPEVQISWFVDGKQMQTAKTQPREEQFNGTYRVVSVLPIGHQDWLKGKQFTCKVNNKALPSP

SEQ ID NO: 12 - protein, 98 aa, Artificial Sequence
  HC-B_D270(A/N)
  VARIANT 37: X = Ala, Asn
MLGGPSVFIFPPKPKDTLLIARTPEVTCVVVDLDPEXPEVQISWFVDGKQMQTAKTQPREEQFNGTYRVVSVLPIGHQDWLKGKQFTCKVNNKALPSP

SEQ ID NO: 13 - protein, 98 aa, Artificial Sequence
  HC-B_D270A
MLGGPSVFIFPPKPKDTLLIARTPEVTCVVVDLDPEAPEVQISWFVDGKQMQTAKTQPREEQFNGTYRVVSVLPIGHQDWLKGKQFTCKVNNKALPSP

SEQ ID NO: 14 - protein, 98 aa, Artificial Sequence
  HC-B_P331(G/A/S)
  VARIANT 98: X = Gly, Ala, Ser
MLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSX

SEQ ID NO: 15 - protein, 98 aa, Artificial Sequence
  HC-B_P331G
MLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSG

SEQ ID NO: 16 - protein, 98 aa, Artificial Sequence
  HC-B_L235(A/Q/V)_S239(A/V)
  VARIANT 2: X = Ala, Gln, Val
  VARIANT 6: X = Ala, Val
MXGGPXVFIFPPKPKDTLLIARTPEVTCVVVDLDPEDPEVQISWFVDGKQMQTAKTQPREEQFNGTYRVVSVLPIGHQDWLKGKQFTCKVNNKALPSP

SEQ ID NO: 17 - protein, 98 aa, Artificial Sequence
  HC-B_L235A_S239A
MAGGPAVFIFPPKPKDTLLIARTPEVTCVVVDLDPEDPEVQISWFVDGKQMQTAKTQPREEQFNGTYRVVSVLPIGHQDWLKGKQFTCKVNNKALPSP

SEQ ID NO: 18 - protein, 98 aa, Artificial Sequence
  HC-B_L235(A/Q/V)_P331(G/A/S)
  VARIANT 2: X = Ala, Gln, Val
  VARIANT 98: X = Gly, Ala, Ser
MXGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSX

SEQ ID NO: 19 - protein, 98 aa, Artificial Sequence
  HC-B_L235A_P331G
MAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSG

SEQ ID NO: 20 - protein, 98 aa, Artificial Sequence
  HC-B_D270(A/N)_P331(G/A/S)
  VARIANT 37: X = Ala, Asn
  VARIANT 98: X = Gly, Ala, Ser
MLGGPSVFIFPPKPKDTLLIARTPEVTCVVVDLDPEXPEVQISWFVDGKQMQTAKTQPREEQFNGTYRVVSVLPIGHQDWLKGKQFTCKVNNKALPSX

SEQ ID NO: 21 - protein, 98 aa, Artificial Sequence
  HC-B_D270A_P331G
MLGGPSVFIFPPKPKDTLLIARTPEVTCVVVDLDPEAPEVQISWFVDGKQMQTAKTQPREEQFNGTYRVVSVLPIGHQDWLKGKQFTCKVNNKALPSG

SEQ ID NO: 22 - protein, 98 aa, Artificial Sequence
  HC-B HC-B_L235(A/Q/V)_S239(A/V)_D270(A/N)
  VARIANT 2: X = Ala, Gln, Val
  VARIANT 6: X = Ala, Val
  VARIANT 37: X = Ala, Asn
MXGGPXVFIFPPKPKDTLLIARTPEVTCVVVDLDPEXPEVQISWFVDGKQMQTAKTQPREEQFNGTYRVVSVLPIGHQDWLKGKQFTCKVNNKALPSP

SEQ ID NO: 23 - protein, 98 aa, Artificial Sequence
  HC-B_L235A_S239A_D270A
MAGGPAVFIFPPKPKDTLLIARTPEVTCVVVDLDPEAPEVQISWFVDGKQMQTAKTQPREEQFNGTYRVVSVLPIGHQDWLKGKQFTCKVNNKALPSP

SEQ ID NO: 24 - protein, 98 aa, Artificial Sequence
  HC-B_L235(A/Q/V)_S239(A/V)_P331(G/A/S)
  VARIANT 2: X = Ala, Gln, Val
  VARIANT 6: X = Ala, Val
  VARIANT 98: X = Gly, Ala, Ser
MXGGPXVFIFPPKPKDTLLIARTPEVTCVVVDLDPEDPEVQISWFVDGKQMQTAKTQPREEQFNGTYRVVSVLPIGHQDWLKGKQFTCKVNNKALPSX

SEQ ID NO: 25 - protein, 98 aa, Artificial Sequence
  HC-B_L235A_S239A_P331G
MAGGPAVFIFPPKPKDTLLIARTPEVTCVVVDLDPEDPEVQISWFVDGKQMQTAKTQPREEQFNGTYRVVSVLPIGHQDWLKGKQFTCKVNNKALPSG

SEQ ID NO: 26 - protein, 98 aa, Artificial Sequence
  HC-B HC-B_L235(A/Q/V)_S239(A/V)_D270(A/N)_P331 (G/A/S)
  VARIANT 2: X = Ala, Gln, Val
  VARIANT 6: X = Ala, Val
  VARIANT 37: X = Ala, Asn
  VARIANT 98: X = Gly, Ala, Ser
MXGGPXVFIFPPKPKDTLLIARTPEVTCVVVDLDPEXPEVQISWFVDGKQMQTAKTQPREEQFNGTYRVVSVLPIGHQDWLKGKQFTCKVNNKALPSX

SEQ ID NO: 27 - protein, 98 aa, Artificial Sequence
  HC-B_L235A_S239A_D270A_P331G
MAGGPAVFIFPPKPKDTLLIARTPEVTCVVVDLDPEAPEVQISWFVDGKQMQTAKTQPREEQFNGTYRVVSVLPIGHQDWLKGKQFTCKVNNKALPSG

SEQ ID NO: 28 - protein, 98 aa, Artificial Sequence
  HC-B_M234(A/Q/V) _L235(A/Q/V)
  VARIANT 1: X = Ala, Gln, Val
  VARIANT 2: X = Ala, Gln, Val
XXGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSP

SEQ ID NO: 29 - protein, 98 aa, Artificial Sequence
  HC-B_M234A_L235A
AAGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSP